(19) United States
(12) Patent Application Publication
(10) Pub. No.: US 2024/0366735 A1
(43) Pub. Date: Nov. 7, 2024

(54) OCULAR ANTIMICROBIAL COMPOSITIONS AND METHODS OF USE THEREOF

(71) Applicant: The University of Hong Kong, Hong Kong (CN)

(72) Inventors: Yau Kei Chan, Hong Kong (CN); Liangyu Zhou, Hong Kong (CN)

(21) Appl. No.: 18/653,590

(22) Filed: May 2, 2024

Related U.S. Application Data
(60) Provisional application No. 63/499,590, filed on May 2, 2023.

(51) Int. Cl. A61K 38/44; A61K 9/00; A61K 38/06; A61K 38/07; A61K 38/08; A61K 41/17; A61P 27/02; A61P 31/04

(52) U.S. Cl. CPC A61K 38/443; A61K 9/0051; A61K 38/06; A61K 38/07; A61K 38/08; A61K 41/17; A61P 27/02; A61P 31/04; C12Y 101/03004; C12Y 113/12004

(57) ABSTRACT

Ocular antimicrobial compositions and methods and method of use thereof are provided. The compositions incorporate photoactive heterojunction materials can be incorporated into a suitable carrier, alone or in combination with an enzyme capable of generating ROS upon contact with the eye for the treatment of ocular conditions. The method includes contacting the eye of a subject in need thereof, the subject has or has been diagnosed with microbial keratitis, with an effective amount of the disclosed composition. The method further includes contacting the composition in the subject's eye with near infrared radiation (NIR).

### CROSS-REFERENCE TO RELATED APPLICATIONS

This application claims benefit of U.S. Provisional Application No. 63/499,590, filed on May 2, 2023, which is hereby incorporated herein by reference in its entirety

### FIELD OF INVENTION

The disclosed invention is generally in the field of antimicrobial hydrogels for use in treating ocular conditions.

### BACKGROUND OF INVENTION

Antibiotic resistance and infections caused by multidrug-resistant bacteria are global health concerns. The diagnosis for microbial keratitis typically requires microbe culture and histological exanimation but can have low sensitivity and be time-consuming, taking on the order of 3-5 days or more. However, pathogens such as Pseudomonas aeruginosa (P. aeruginosa) can invade the cornea and cause severe corneal opacity in 1-2 days or less. Before pathogens are identified, broad-spectrum, empirical antibiotics such as levofloxacin and ceftazidime are frequently used as first-line treatments. However, use of antibiotics can induce severe adverse effects, including corneal opacification, allergy, and toxicity of the corneal tissues, and excessive use of antibiotics can lead to the development of antimicrobial resistance (AMR). Antibiotics are applied in clinical treatment for microbial keratitis. These complications have promoted innovation of alternatives for antibiotic treatments.

Reactive oxygen species (ROS)-mediated antimicrobial therapies has emerged as a promising strategy to fight against antimicrobial resistance. Through mechanisms of directive DNA damage and membrane lipid peroxidation of bacteria, ROS eradicates bacteria without leading to antimicrobial resistance. For example, one of the photoactivated chromophores for keratitis-corneal cross-linking (PACK-CXL), riboflavin, has been applied clinically to treat microbial keratitis. Riboflavin releases ROS to damage bacterial cells under UV light irradiation. However, riboflavin can penetrate into cornea and undesirably crosslink the collagen component in the corneal tissue. Also, tissue damage facilitated by UV light is a concern. Thus, there remains a need for a need for effective and safer antibiotic-free treatment strategies for microbial infections of the eye.

It is an object of the present invention to provide compositions to treat and/or prevent microbial ocular conditions.

It is another object of the invention to provide a method of making the antimicrobial heterojunction hydrogel compositions.

It is also an object of the present invention to provide compositions with antimicrobial activity, which can be used to combat drug resistant microbial infections.

### SUMMARY OF THE INVENTION

The disclosed compositions and methods are based at least on the discovery that photoactive heterojunction materials can be incorporated into a suitable carrier and used alone or in combination with an enzyme capable of generating ROS upon contact with the eye for the treatment of ocular conditions. Thus, compositions, methods of making the compositions and methods of using the compositions to treat and/or prevent microbial ocular conditions are provided.

The disclosed antimicrobial ocular compositions preferably include a hydrogel material as the carrier, one or more photoactive heterojunction nanomaterials and an ROS-generating/oxidoreductive enzyme. The antimicrobial composition is a tear glucose-responsive and photoactive antimicrobial hydrogel, preferably, a hydrogel bandage (tgr-PAHB). The tgr-PAHB release reactive oxygen species, in an amount effective to kill pathogens in the presence of near infra-red (NIR) irradiation and eye tears. The antimicrobial hydrogel compositions are designed to provide synergistic reactive oxygen species (ROS)-induced photodynamic therapy (PDT), chemodynamic therapy (CDT), and hyperthermia-induced photothermal therapy (PTT). The disclosed antimicrobial ocular compositions generate an effective amount of ROS to treat microbial infections and reducing virulence of bacteria associated with ocular conditions, following contact with eye tears and NIR. Preferably, the photoactive heterojunction materials are formed of graphene oxide (Go) and Copper ferrite nanoparticles (CFN) and are immobilized in the hydrogel carrier material. The photoactive heterojunction materials produce ROS and heat under NIR irradiation, thereby damaging the DNA and cell membrane of bacteria. Preferably, the ROS-generating/oxidoreductive enzyme is glucose oxidase (GOx) and/or the hydrogel is formed from one or more polymers preferably, poly-epsilon-lysine (pεK). The amount of photoactive materials that can be incorporated into the hydrogel range from about 50 μg to about 250 μg, preferably from about 80 μg to about 200 μg, for example, about 30, 40, 50, 60, 70, 80 or 90 μg, etc. The amount of ROS-generating/oxidoreductive enzyme such as glucose oxidase can be from about 10 to about 100 μg, for example, about 20, 30, 40, 50, 60, 70, 80 or 90 μg, including all intervening values.

Generally, the ROS-generating/oxidoreductive enzyme is immobilized on the carrier material such as a hydrogel base, allowing it to catalyze glucose in tears into hydrogen oxide (OH·). The heterojunction materials can utilize OH·, thereby generating ROS to kill bacteria. The consumption of glucose further reduces the glucose supply in the surrounding environment and may hinder the growth of bacteria.

Also disclosed are methods of treating ocular conditions using the disclosed antimicrobial hydrogel compositions. The method includes contacting the eye of a subject in need thereof, with an effective amount of the disclosed composition. The method further includes contacting the composition in the subject's eye with near infrared radiation (NIR). In a preferred embodiment, the subject has or has been diagnosed with microbial keratitis, and the composition is applied to the infected site. The NIR can be provided by a NIR light source machine which includes a probe that can irradiate NIR laser beam; i.e., a portable probe, connected to a light source (with a picture), for example, an 808 nm light source can be used in the disclosed methods. The probe is aligned to the tgr-PAHB to deliver the treatment. In some forms, the light source is a fixed instrument in a clinic. In some forms, the light source is a transportable device e.g., a handheld LED Infrared Light Therapy Pen®. In some forms, the distance for treatment is approximately 3-4 cm. In some forms, the duration of treatment is from about 20 minutes to about 40 minutes daily for about 1 to 10 for example, 2, 3, 4, 5, 6, 7, 8, 9 or 10 days, preferably, between about 3 days to about 5 days.

### BRIEF DESCRIPTION OF DRAWINGS

FIGS. 1A-1F show the material characterizations of various samples. FIG. 1A and 1B show the morphology of photosensitive heterojunction GO/CFN characterization: (FIG. 1A) TEM images, (FIG. 1B) SEM images, and chemical structures: (FIG. 1C) XRD analysis. FIGS. 1D-1H show the morphology of tgr-PAHB characterization: (FIG. 1D) SEM images, and chemical structures: (FIG. 1E) FT-IR spectra, (FIG. 1F) BCA protein assay measurement,.

FIGS. 2A-2F. Real-time photothermal monitoring of various samples, showing the photothermal property of tgr-PAHB. Photothermal heating curves of (FIG. 2A) GO, CFN, GO/CFN; (FIG. 2B) GO/CFN with varying NIR density, (FIG. 2C) varying concentration go GO/CFN. (FIG. 2D) Real-time thermal images, (FIG. 2E) heating curves and (FIG. 2F) heating and cooling circles of tgr-PAHB.

FIGS. 3A-3L. Measurement of photodynamic and chemodynamic properties of. various samples. FIG. 3A shows the mechanism MB oxidation by hydroxyl radicals, (FIG. 3B) oxidation of MB by HB with varying time, (FIG. 3C) oxidation of MB by tgr-PAHB with varying time, (FIG. 3D) oxidation of MB by tgr-PAHB with or without NIR. (FIG. 3E) mechanism DPBF oxidation by singlet oxygen, (FIG. 3F) oxidation of DPBF by HB with varying time, (FIG. 3) oxidation of DPBF by tgr-PAHB with varying time, (FIG. 3H) oxidation of DPBF by tgr-PAHB with or without NIR. (FIG. 3I) mechanism TMB oxidation by hydroxyl radical produced through chemodynamic reactions, (FIG. 3J) oxidation of TMB by samples with H2O2 addition in darkness, (FIG. 3K) oxidation of TMB by samples with glucose addition in darkness. (FIG. 3L) ESR spectra of ·OH and ¹O₂ with a magnetic field intensity from 318 to 328 mT.

FIGS. 4A-4C show the antibacterial performance for corneal bandages in vitro: (FIG. 4A) bacterial efficiency against bacteria; (FIG. 4B) SEM morphologies of bacteria treated by different samples (the red arrows indicate the shrinking membranes, while the red area indicates leakage of bacteria). (FIG. 4C) The intracellular ROS level of S. aureus, P. aeruginosa.

FIGS. 5A-5B show antibiofilm experiments. FIG. 5A. Quantitative analysis destroyed biofilm through measure with crystal violet. FIG. 5B SEM images of matured biofilm and bacteria after treated with samples.

FIGS. 6A-6H show biocompatibility of tgr-PAHB: (FIG. 6A) Schematics indicate the induce of injury wound on epithelium; (FIG. 6B) the cell viability measure with cck-8 after treated with samples; (FIG. 6C) the cell viability of HCE-T cells after daily treatment with tgr-PAHB; (FIG. 6D) The defect area of the injury after daily treatment. (FIG. 6E) wound healing rate of epithelium after treated with samples. (FIG. 6F) Schematics indicate the induce of scratch line on epithelium. (FIG. 6G) images and (FIG. 6H) the quantification of scratch line on epithelium after treated with samples.

FIGS. 7A-7F show treatment efficacy of tgr-PAHB compared to clinical applied antibiotics for keratitis treatment on ex-vivo pig eyes model: FIG. 7A. Schematic diagram of establishment of infected porcine cornea and treatment with tgr-PAHB; FIG. 7B. Photothermal effect of tgr-PAHB on isolated porcine cornea; FIG. 7C. Images of MRSA-induced ex-vivo model and quantification of MRSA from homogenized cornea; (FIG. 7D) images of MDRPA-induced ex-vivo model and quantification of MDR-PA from homogenized cornea. (FIG. 7E) SEM images of MRSA or MDR-PA infected cornea with varying treatment. (FIG. 7F) shows isolated bacteria from the collected tissue from rabbit eyes. Control=the positive control group; treated=the treatment group

FIGS. 8A-8B show an exemplary NIR laser probe (FIG. 8A) and laser source (FIG. 8B). FIGS. 9A and 9B are schematic illustrations of (FIG. 9A) the preparation of tgr-PAHB and (FIG. 9B) the mechanisms of pathogens eradication on ocular surface by tgr-PAHB during BK treatment. FIG. 9C shows Hydrogelators 1-10 derived from NaA-capped cationic peptide mimics. FIG. 9D discloses Structure of peptide mimic hydrogelators. (FIGS. 9C and 9D are reproduced from Scientific Reports volume 12, Article number: 22259 (2022)).

FIG. 10A are AFM images of Go/CFN heterojunction. FIG. 10B is a photo showing the transparency of the hydrogel bandage. FIG. 10C are graphs showing antibacterial efficiency after immobilization with GOx. FIG. 10D is a bar graph showing the remaining S. aureus (S.A.) and P. aeruginosa (P.A.) after treated by antibacterial contact lens (150 μg), with different exposure time; Go to CFN (1:10), 808 nm NIR (1 W/cm2). FIG. 10E is a bar graph showing the zetapotential. FIG. 10F details the contact angle and water absorption.

FIG. 11 shows the intensity of transmitted NIR light spot after applying hydrogel or tgr-PAHB.

FIG. 12 shows antibacterial properties of the exemplary heterojunction hydrogels against S. aureus and P. aeruginosa.

FIG. 13A is a bar graph showing the cell viability and cell morphology of 3T3 cells after 6 h incubation with Go/CFN Pek leachate. Scale bar: 100 μm. FIG. 13B shows the consumption of GSH (anti-oxide molecules that act as a defending reagent presenting in bacteria) in solution, tgr-PAHB can inactivate GSH quickly. FIG. 13C illustrates the leakage of bacterial cellular contents after treated by tgr-PAHB, which suggests that treatment with tgr-PAHB can induce damage to bacteria membrane and cause bacterial contents leakage.

FIG. 14 are representative images of the ROS levels in treated S. aureus and P. aeruginosa bacteria.

FIG. 15 is a schematic of ROS generation mechanisms.

FIG. 16 is a schematic of the photocatalytic antibacterial mechanism.

### DETAILED DESCRIPTION OF THE INVENTION

### I. Definitions

“Heterojunction” generally refers to the interface between two different semiconductors with unequal band structure, which can result in band alignments.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied.

“Active Agent,” as used herein, refers to a physiologically or pharmacologically active substance that acts locally and/or systemically in the body. An active agent is a substance that is administered to a patient for the treatment (e.g., therapeutic agent), prevention (e.g., prophylactic agent), or diagnosis (e.g., diagnostic agent) of a disease or disorder. “Ophthalmic Drug” or “Ophthalmic Active Agent”, as used herein, refers to an agent that is administered to a patient to alleviate, delay onset of, or prevent one or more symptoms of a disease or disorder of the eye, or diagnostic agent useful for imaging or otherwise assessing the eye.

“Effective amount”, in reference to an active agent such as a self-assembling peptide or biomolecule, pharmaceutical agent, etc., refers to the amount necessary to elicit a desired biological response. As will be appreciated by those of ordinary skill in this art, the effective amount of an agent may vary depending on such factors as the desired biological endpoint, the agent to be delivered, the nature of the site to which the agent is delivered, the nature of the conditions for which the agent is administered, etc. For example, the effective amount of a composition for treatment of a disease or disorder may be an amount sufficient to promote recovery to a greater extent than would occur in the absence of the composition.

“Preventing” refers to causing a condition, state, disease, symptom or manifestation of such, or worsening of the severity of such. Preventing includes reducing the risk that a condition, state, or disease, or symptom or manifestation of such, or worsening of the severity of such, will occur.

The terms “treat”, “treatment” and “treating” refer to the reduction or amelioration of the progression, severity and/or duration of one or more symptoms of an injury, disease or disorder, delay of the onset of a disease or disorder, or the amelioration of one or more consequences, indications or symptoms (preferably, one or more discernible symptoms) of an injury, disease or disorder, resulting from the administration of one or more therapies (e.g., one or more therapeutic agents such as a compound as described). The terms “treat”, “treatment”, and “treating” also encompass the reduction of the risk of developing a disease or disorder, and the delay or inhibition of the recurrence of a disease or disorder.

The terms “increase,” “enhance,” “stimulate,” and/or “induce” (and like terms) generally refer to the act of improving or increasing, either directly or indirectly, a function or behavior relative to the natural, expected, or average or relative to current conditions. For instance, something that increases, stimulates, induces or enhances anti-inflammatory effects might induce the production, and/or secretion of anti-inflammatory cytokines, and/or infiltration of immune cells that mediate anti-inflammatory responses, such as Treg, or Th17 cells.

The term “subject” or “individual” includes both humans, mammals (e.g., cats, dogs, horses, etc.), and other living species that are in need of treatment for inflammatory and/or autoimmune conditions/diseases.

The term “carrier” or “excipient” refers to an organic or inorganic, natural or synthetic inactive ingredient in a formulation, with which one or more active ingredients are combined. In some embodiments, a carrier or an excipient is an inert substance added to a pharmaceutical composition to further facilitate administration of a compound, and/or does not cause significant irritation to an organism and does not abrogate the biological activity and properties of the administered compound.

### II. Compositions

A. Antimicrobial ROS-Generating Hydrogel Compositions

Compositions containing a photoactive heterojunction material and ROS generating enzymes in a carrier material such as a hydrogel, are disclosed for the treatment of ocular conditions. Thus, in certain embodiments, disclosed herein is a therapeutically effective hydrogel composition for treating ocular conditions.

The disclosed compositions are activated to produce ROS in response to a light source and upon coming into contact with a substrate for the ROS generating enzyme. The disclosed compositions therefore rely on a combination of ROS) induced photodynamic therapy (PDT), chemodynamic therapy (CDT) and hyperthermia-induced photothermal therapy (PTT) for the treatment of ocular conditions. The disclosed antimicrobial heterojunction compositions are tear glucose-responsive and photoactive, and can release ROS, and eradicate pathogens in the presence of near infra-red (NIR) irradiation and eye tear.

ROS-based antibacterial strategies, antibacterial dynamic therapy (ADT) can be mainly divided into endogenous and exogenous. Endogenous ADT refers to chemodynamic therapy (CDT), through which materials catalyze hydrogen peroxide (H₂O₂) to produce hydroxyl radicals (·OH) through Fenton(-like) reactions. The Fenton(-like) reactions are triggered by Fe(III)/Fe(II) or Cu(II)/Cu(I) redox cycling. Immobilizing enzymes which can catalyze glucose or lactic acid increases the amount production of H₂O₂, subsequently improves the efficiency of eradication against bacteria on infectious site. In contrast, materials in exogenous ADT produce ROS under the excitation of external energy such as light or ultrasound. The process that photosensitive materials eliminate bacteria through releasing ROS under light irradiation is named as photodynamic therapy (PDT). By selecting semiconductors in bio-heterojunction materials such as carbon-related materials (graphene, graphene oxide) which can generate a vast amount of heat under NIR irradiation, the disclosed compositions can additionally provide hyperthermia-induced photothermal therapy (PTT). Excessive heat allows the material to destroy bacteria through hyperthermia-induced photothermal therapy (PTT). Furthermore, the heat generated increases the antibacterial efficiency of PDT by facilitating the release and diffusion of ROS.
1. Photoactive Heterojunction Material

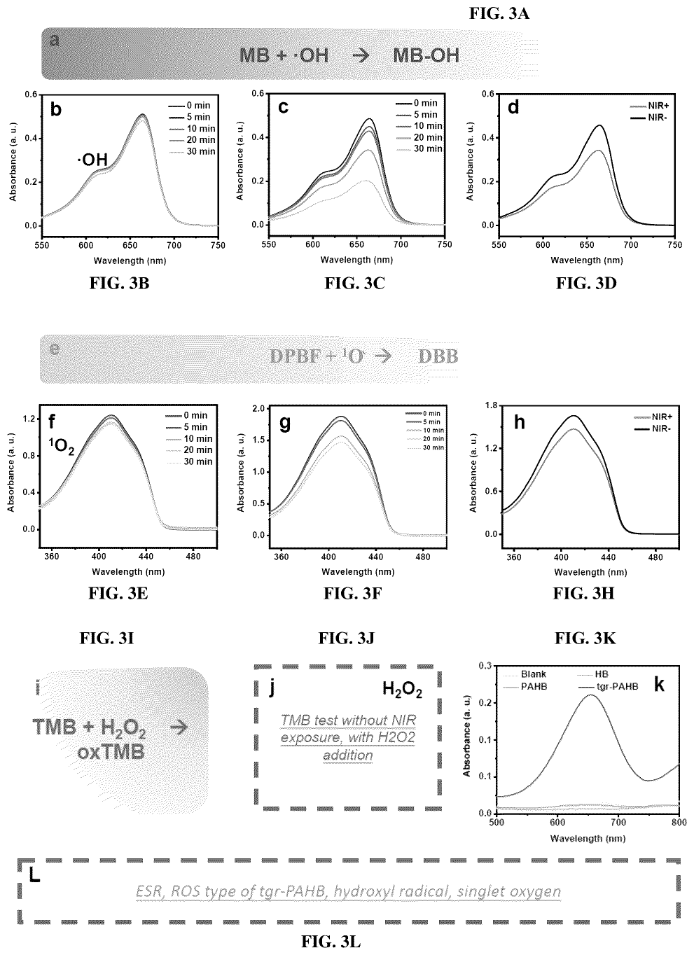

The disclosed antimicrobial hydrogel compositions typically contain one or more photoactive heterojunction materials. Photoactive heterojunction materials are known in the art and include, but are not limited to materials containing titanium dioxide (TiO₂), copper (Cu₂), and Zinc oxide (ZnO). Heterojunction photocatalysts/photoactive heterojunction materials and methods of making the same, are known in the art (reviewed in Mutalik et al. Nanomaterials, Vol. 10(6): 643 (2020), doi: 10.3390/nano 10040643 and Zhou et al., ACS Appl. Bio. Mater., Vol. 4, pages 3909-3936 (2021) and Low, et al., (2017). Heterojunction photocatalysts. Advanced materials, 29 (20), 1601694.). A heterojunction, in general, is defined as the interface between two different semiconductors with unequal band structure, which can result in band alignments. Typically, there are three types of conventional heterojunction photocatalysts, those with a straddling gap (type-I), those with a staggered gap (type-II), and those with a broken gap (type-III) (see FIG. 3 in Low, et al., (2017). Heterojunction photocatalysts. Advanced materials, 29(20), 1601694.).

Exemplary combinations of photoactive heterojunction materials include but are not limited to Graphene oxide/copper ferrite nanoparticles (Go/CFN), Silver-titanium oxide (Ag—TiO₂), TiO₂/AgVO₃, Ag/TiO₂/cellulose, Alginate/Au—TiO₂, Lithium-titanate in the low-density polyethylene matrix (Li—TiO₂/LDPE), Cu—TiO₂, TiO₂/—Fe₂O₃, TiO2 doped with Bi (Bi—TiO2 group) and TiO₂ co-doped with urea and Bi (Urea, Bi—TiO₂ group; U,Bi—TiO₂), chitosan films containing melon/TiO₂ (CTS/MTiO₂), TiO₂ nanoparticles and graphene sheets (TiO₂/GSs), graphene oxide and cuprous oxide (rGO—Cu₂O), and Zinc oxide-selenium (ZnO—Se). The amount of photoactive materials that can be incorporated into the hydrogel range from about 50 μg to about 250 μg, preferably from about 80 μg to about 200 μg.

In preferred embodiments, the photoactive heterojunction material is formed of graphene oxide (Go) and Copper ferrite nanoparticles (CFN). Bio-heterojunction materials of copper ferrite nanoparticle (CFN) and graphene oxide (GO) can destroy pathogenic bacteria through synergistic strategy of PDT and PTT. Accompanied by multivalent copper (Cu) and iron (Fe), copper ferrite nanoparticles (CFN, CuFe2O4), possess excellent NIR absorbance for photothermal effect and Fenton(-like) reaction activity. GO as a class of two-dimensional biocompatible material, provides binding sites to CFN for formation of hetero-interface. After hybridization into GO/CFN bio-heterojunction, the hetero-interface between GO and CFN can produce singlet oxygen (¹O₂) under NIR exposure to eradicate pathogenic bacteria through PDT. Meanwhile, the released copper ions can improve corneal re-epithelialization and promote the process of wound healing.

Methods of making photoactive heterojunction materials are known. For example, heterojunction material formed of graphene oxide (Go) and Copper ferrite nanoparticles (CFN) (Go/CFN) can be synthesized via a hydrothermal reaction. As described in the non-limiting Examples, a desired amount of Go nanosheets are dissolved ethylene glycol. Then, an amount of iron chloride hexahydrate, copper chloride dihydrate, sodium acetate, and polyvinylpyrrolidone are added to the Go-ethylene glycol solution. The mixture is then ultrasonicated, and transferred to a Teflon autoclave. The hydrothermal reaction is kept at a suitable temperature for a pre-determined amount of time e.g., 180° C. for 24 hours. Finally, the Go/CFN heterojunction powders is collected, rinsed using ethyl-alcohol and deionized (D.I.) water and dried.

The antimicrobial activity produced from the inclusion of photoactive heterojunction materials in the antimicrobial hydrogel composition depends on the principle of photocatalysis.

Photocatalysis is mainly a process in which the electrons generated from photosensitizer (PS) are excited by light, an excited state with the subsequent redox reactions with the reactants (Reviewed in Zhou et al., ACS Appl. Bio. Mater., Vol. 4, pages 3909-3936 (2021)). The photocatalytic antibacterial process is the reaction between bacteria and ROS. As an intermediate product, ROS mainly results from an incomplete reduction reaction between light-excited electrons/holes and oxygen-(O₂) or water during the photocatalytic reaction process, which exhibits higher activity (Jia, et al. Rejuvenated Photodynamic Therapy for Bacterial Infections. Adv. Healthcare Mater. 2019, 8, 1900608; Wang, Y. et al. Construction of nanomaterials with targeting phototherapy properties to inhibit resistant bacteria and biofilm infections. Chem. Eng. J. 2019, 358, 74-90). ROS mainly include singlet oxygen (¹O₂), superoxide anions (·O₂—), hydroxyl radicals (·OH), etc. (Wang, L et al. Using porous magnetic iron oxide nanomaterials as a facile photoporation nanoplatform for macromolecular delivery. J. Mater. Chem. B 2018, 6, 4427-4436). For example, in an embodiment incorporating the metal oxide TiO₂, when the energy of light is higher than the band gap of TiO₂, the electrons can jump from a valence band (VB) to a conduction band (CB), forming electron-hole pairs (Mao, et al. Repeatable photodynamic therapy with triggered signaling pathways of fibroblast cell proliferation and differentiation to promote bacteria-accompanied wound healing. ACS Nano 2018, 12, 1747-1759). Holes can interact with OH— or H₂O to generate ·OH. The ·OH is a highly active species that can oxidize a variety of organic substances indiscriminately. Photogenerated electrons can also interact with O₂ to generate other ROS such as ·O₂—, and these reactive oxygen radicals can also participate in redox reactions. The reaction process is shown in the following equations:

TiO₂+hv→TiO₂(e⁻, h⁺) (1)

e⁻+h⁺→heat or hv (2)

h⁺+OH_{ads}⁻→HO* (3)

h⁺+H₂O_{ads}→HO*+H⁺ (4)

e⁻+O₂→O₂^{-*} (5)

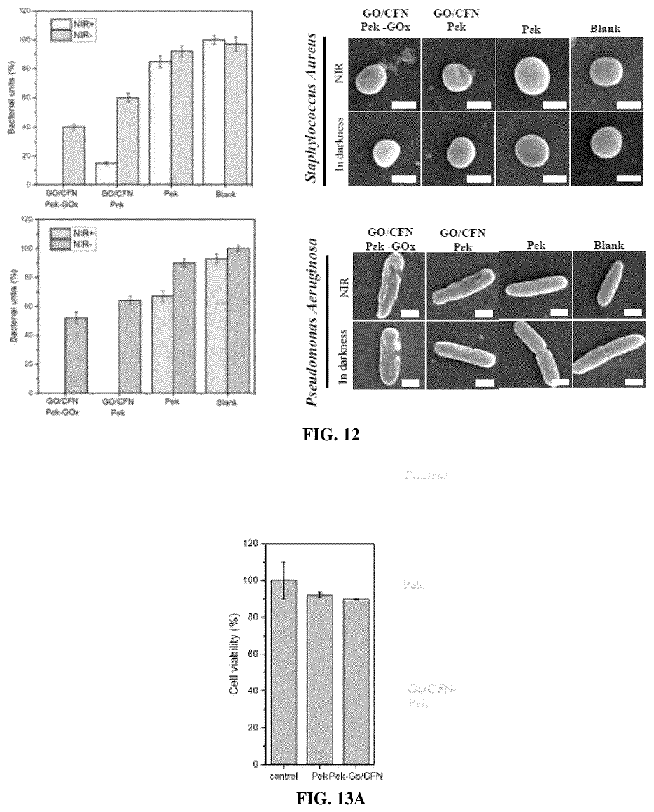

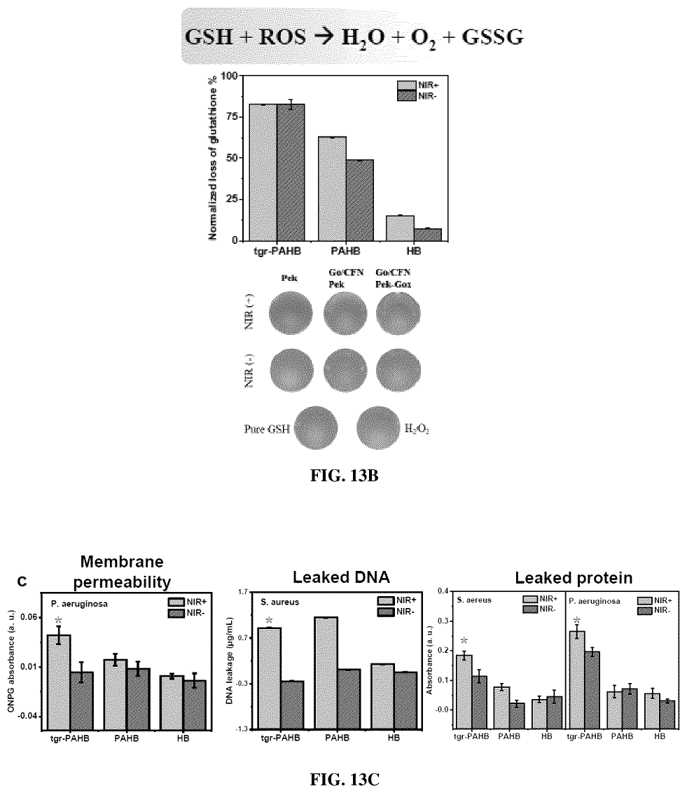

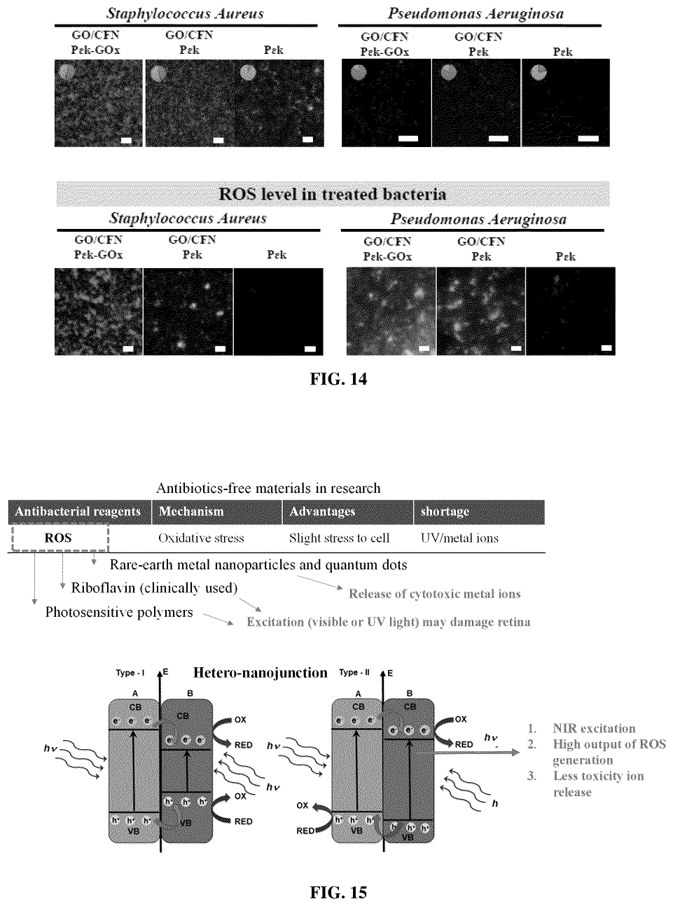

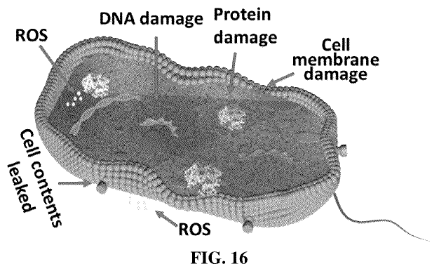

Thus, photocatalytic antibacterial therapy depends a photosensitive reaction accompanied by subsequent biological effects. This process is that a specific wavelength of laser radiation excites a photosensitizer (PS) at the bacterial infected sites, and the produced photogenerated electrons or holes react with surrounding oxygen or water to generate highly active oxygen radicals (Sun, J. et al. Synergistic photodynamic and photothermal antibacterial nanocomposite membrane triggered by single NIR light source. ACS Appl. Mater. Interfaces 2019, 11, 26581-26589). As shown in FIGS. 12 and 13, the ROS react with the cell membrane or go into the bacteria, thus destroying bacterial cell membrane, inducing the leakage of the inside substance, and further inactivating bacterial DNA and proteins, thereby killing bacteria (Kim, S. et al. Functional manganese dioxide nanosheet for targeted photodynamic therapy and bioimaging in vitro and in vivo. 2D Mater. 2017, 4, 025069). ROS can cause oxidative stress within bacteria, and extracellular ROS may enter into the bacteria and cause dramatically boosted ROS levels intracellularly, which would react with antioxidative enzymes, disrupting the balance of oxidation and antioxidation in bacterial cells.
2. Carrier Material

A preferred carrier material is hydrogel-based. The utilization of a hydrogel, preferably, PεK hydrogel provides anchors for the heterojunction material such as GO/CFN and the ROS generating enzyme, for example, Gox, which avoids dilution clearance of heterojunction material and ROS generating enzyme (e.g., GO/CFN and Gox) by eye tear reflux when applied topically during the treatment. GOx on tgr-PAHB catalyzes eye tear glucose into H₂O₂ and suppresses the growth of bacteria. Then, GO/CFN heterojunction in tgr-PAHB produces ROS through H₂O₂ enhanced CDT and synergistic PDT/PTT modalities.

However, any material useful for ocular applications and which includes a matrix within which the heterojunction nanomaterials and ROS/oxidoreductase enzyme can be immobilized, are also useful in the disclosed antimicrobial compositions.

Hydrogels are three-dimensional (3D) cross-linked polymer networks, which can absorb and retain large amount of water. A hydrogel is a three-dimensional (3D) network of hydrophilic polymers that can swell in water and hold a large amount of water while maintaining the structure due to chemical or physical cross-linking of individual polymer chains. Hydrogels are mainly formed from biopolymers and/or polyelectrolytes. Hydrogels useful in the disclosed composition can be made using any polymeric material that is suitable for ocular applications Such materials used for the preparation of antimicrobial hydrogels are known in the art and include for example, polymers with quaternary ammonium functional groups, natural peptides such as collagen, gelatine, polysaccharides such as starch, alginate, and agarose, phospho- and sulfo-derived polymers, phenol and benzoic acid derived polymers, and polyelectrolyte multilayers (Ahmed, J Adv Res. 2015 March; 6 (2): 105-121; Pierau and Versace, Materials (Basel), 14 (4): 787 (2021)). Other examples include covalently crosslinked hyaluronic acid, and the hyaluronic acid may be modified or unmodified. Unmodified hyaluronic acid may be covalently crosslinked by a variety of methods, including crosslinking using 1,4-butanediol diglycidyl ether (BDDE), divinylsulfone, and dihydrazide. The hyaluronic acid may be modified to change the charge of the molecule, change its biological activity, or to include groups that may be used for crosslinking purposes. Particularly useful are thiolated hyaluronic acid or thiolated carboxymethyl hyaluronic acid. Modified hyaluronic acid may be crosslinked with an external molecule for crosslinking, or without an external crosslinker molecule. For crosslinking thiolated HA or CMHA, a molecule with thiol-reactive sites, such as acrylates, methacrylates, haloacetates, haloacetamides, or maleimides, may be used as an external crosslinker molecule, examples of which include poly (ethylene glycol) diacrylate and poly (ethylene glycol) bisbromoacetate. For crosslinking without an external crosslinker molecule, in particular, thiolated HA or CMHA may be disulfide crosslinked via an oxidation process. Such disulfide crosslinking may be aided by use of an oxidant such as sodium hypochlorite or peroxide.

When modified HA is crosslinked via the modification (e.g., disulfide crosslinking of thiolated HA), the level of modification may be adjusted to control the amount of crosslinking of the hydrogel, such that a higher level of modification leads to more crosslinking. Particularly useful for formulating hydrogels of the present disclosure is thiolated HA or thiolated CMHA, where the thiol modification is about 0.05 to about 1.0 μmol thiol per mg of HA or CMHA. Modification levels within this range are particularly suitable for forming crosslinked hydrogels with a desired shear-thinning profile and viscosity.

When placed on the surface of the eye, shear-thinning hydrogels made using thiolated CMHA and having a concentration range of about 3 to about 10 mg/ml remain in contact with the eye surface for at least 30 minutes and up to about 2 hours. As used herein, the term “shear-thinning” refers to a state in which viscosity decreases as shear rate increases, thereby indicating shear-thinning behavior.

In a preferred embodiment, the polymer used to form the hydrogel base is poly-epsilon-lysine (PεK), resulting in a hydrogel with intrinsic antibacterial activity. Poly-epsilon-lysine (pεK) is a cationic peptide with intrinsic antimicrobial properties and broad-spectrum antimicrobial activity against gram-positive and-negative bacteria, yeasts, and fungi. ε-polylysine is a kind of natural polymeric material forming hydrogel, it is obtained by microbial fermentation. The molecular weight of ε-polylysine is between 3 kDa and 5 kDa. A ε-polylysine hydrogel, it has the following constitutional unit:

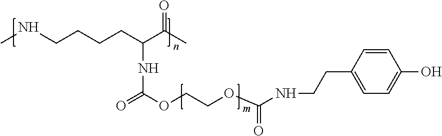

wherein, n is any natural number of 20 to 30, m is any natural number of 50 to 70. Epsilon-polylysine is available from BOC Sciences, Catalog NO.: B0001-011213 CAS NO.: 28211 Apr. 3. Methods for making PεK hydrogels, are known in the art (Aveyard et al. ACS Appl Mater Interfaces 2019;11 (41): 37491-50).

Other hydrogels with intrinsic antibacterial activity include Fmoc-diphenylalanine and Fmoc-capped short peptides bearing either lysine-rich or pyridinium groups (have been found with moderate antibacterial activities against Gram-positive and Gram-negative bacteria); anthranilamide-based diphenylalanine peptide mimics formed self-assembled hydrogels; hydrogels prepared by self-assembly of naphthyl anthranilamide (NaA) capped amino acid based cationic peptide mimics (Reviewed in Aldilla, et al., Scientific Reports volume 12, Article number: 22259 (2022)).

For example, McCloskey et al discloses Fmoc-peptides (FmocFF (a), FmocFFKK (b), Fmoc-FFFKK (c) and FmoCFFOO (d), shown below) which formed surfactant-like soft gels at concentrations of 1% w/v.

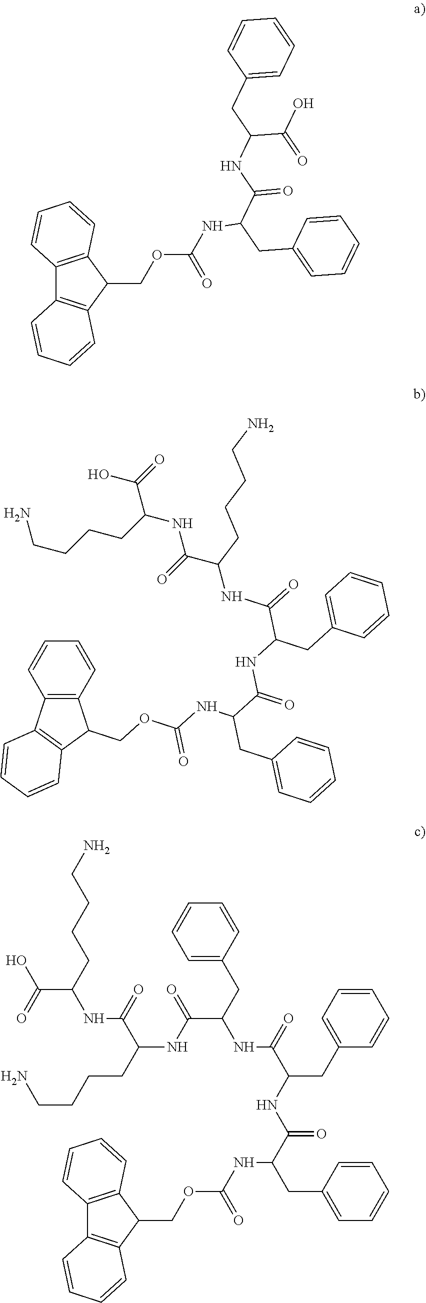 [SMILES: O=C(NC(Cc1ccccc1)C(=O)NC(Cc1ccccc1)C(=O)O)OCC1c2ccccc2-c2ccccc21 ; NCCCCC(NC(=O)C(CCCCN)NC(=O)C(Cc1ccccc1)NC(=O)C(Cc1ccccc1)NC(=O)OCC1c2ccccc2-c2ccccc21)C(=O)O ; NCCCCC(NC(=O)C(CCCCN)NC(=O)C(Cc1ccccc1)NC(=O)C(Cc1ccccc1)NC(=O)C(Cc1ccccc1)NC(=O)OCC1c2ccccc2-c2ccccc21)C(=O)O]

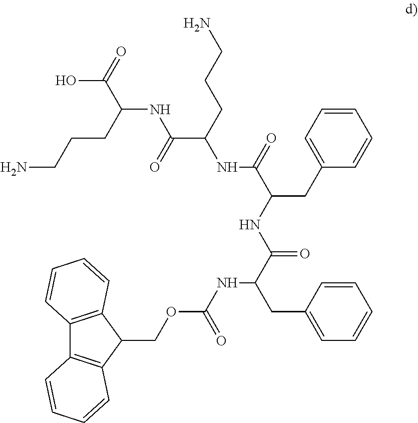 [SMILES: NCCCC(NC(=O)C(CCCN)NC(=O)C(Cc1ccccc1)NC(=O)C(Cc1ccccc1)NC(=O)OCC1c2ccccc2-c2ccccc21)C(=O)O]

(McCloskey, A. P., Draper, E. R., Gilmore, B. F. & Laverty, G. Ultrashort self-assembling Fmoc-peptide gelators for anti-infective biomaterial applications. J. Pept. Sci. 23, 131-140 (2017)). Other exemplary peptides are disclosed in Debnath, et al., Phys. Chem. B 2010, 114, 13, 4407-4415.

3. ROS Generating/Oxidoreductase Enzymes

Generally, the enzymes incorporated in the hydrogel base are glucose-sensitive and generate one or more reactive oxygen species (ROS) upon reaction with the glucose in eye tears (See FIG. 9B). The amount of enzyme GOx can be varied from about 20 to about 100 μg, for example, about 30, 40, 50, 60, 70, 80, or 90 μg. Exemplary reactive oxygen species includes but are not limited to hydrogen peroxidases (H₂O₂ (H₂O₂), superoxide anion (O₂⁻), hydroxyl radical (·OH), and the singlet oxygen (¹O₂).

An exemplary reaction between the oxidative enzymes and the glucose in tears is shown below:

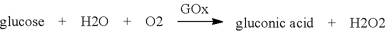

Preferably, the oxidoreductive enzyme suspended in the enzyme-modified hydrogel is glucose oxidase (GOx). The microenvironment on infected ocular surface consists of precorneal tear film with a thickness of 3 μm.^{39 40} Normally, glucose in tear film promotes the growth of pathogenic bacterial, which further aggravates the infection on cornea. The oxidative enzyme of glucose, Glucose oxidase (GOx), can suppress the bacterial growth and produce H₂O₂ by consuming glucose from around the infectious microenvironment. Meanwhile, H₂O₂ produced by GOx facilitates the production of highly oxidative ·OH with the presence of GO/CFN through the Fenton(-like) reaction.

The ROS generating enzyme can in some forms, be lactic oxidase. Lactate oxidase (LOx) can be used to convert L-lactate and oxygen into pyruvate and hydrogen peroxide. Lactate is a main component in basal tears, and it mainly comes from the corneal epithelium.

The disclosed compositions (which include a hydrogel incorporating photoactive heterojunction materials and ROS generating/oxidoreductase enzyme) can be produced using any suitable techniques, which ensure incorporation of the components into the hydrogel, such that the ROS generating/oxidoreductase enzyme can come in contacts with and catalyze its substrate. Many techniques, reactive groups, chemistries, etc. for linking components of the disclosed enzyme-modified hydrogel base are known and can be used with the disclosed components and compositions. For example, peptide crosslinkers that can be used to crosslink other molecules, elements, moieties, etc. to the disclosed compositions, surface molecules, peptides, internalization elements, tissue penetration elements, cargo compositions are known in the art and are defined based on utility and structure. In preferred embodiments, the crosslinker is azelaic acid. In some forms, one or more reagents may be added to help the crosslinker to dissolve. Additional reagents include but are not limited to 4-methyl morpholine, polyvinyl pyrolidine (PVP), and Di-carboxylic acids can be applied as the cross linker in preparing the hydrogel, including hexanedioic, octanedioic, nonanedioic, decanedioic

The disclosed compositions can include other agents, such as therapeutic, permeation enhancers, prophylactic, or diagnostic agents. Permeation enhancers include, but are not limited to, lysophosphatidilo lipids (lysophosphatidylcholine, LPC), calcium chelators (EDTA), benzalkonium chloride, cetylpyridinium chloride, palmitoyl carnitine, non-ionic surfactants (Brij 35, Brij 78, Brij 98, sodium deoxycholate, poly oxyethylene-9-lauryl ether), surface-active heteroglycosides and bile salts (deoxycholate, taurodeoxycholate, and glycocholate), and glycosides (saponins, digitonin, caprylic acid, capric acid).

An exemplary method of preparing the disclosed Go/CFN PεK-Gox heterojunction hydrogels is described in the non-limiting Examples. Briefly, solution A is prepared by dissolving 0.1485 g Pεk and 0.0488 g Azelaic acid in 0.66 mL DI-water with addition of polyvinyl pyrrolidone (PVP) and 4-Methylmorpholine (NMM). Solution B is prepared by dissolving 0.05 g N-hydroxysuccinimide, and 0.2481 g 1-(3-Dimethylaminoproyl)-3-ethylcarbodiimide HCl in 0.33 mL DI-water and immediately transferring to an ice bath. The synthesized Go/CFN and Glucose oxidase can then be dissolved in 5% PVP to prepare 8 mg/mL heterojunction solution and 4 mg/mL Gox solution respectively. 1.23 mL of 8 mg/mL Go/CFN and 440 μL 4 mg/mL Gox is then added to the mixture of solution A and B. The biocidal hydrogel bandage is obtained via polymerization overnight and washed with DI-water and 10% NMM to remove unreacted reagents. Since it is possible the —COOH groups on GOx to react with —NH2 groups on Pεk during the polymerization process, the GOx can be fixed onto the hydrogel.

B. Pharmaceutical Compositions

The disclosed antimicrobial hydrogel compositions can be provided dosage forms that are suitable for ocular application, including, but not limited to an ophtlamic gel (including contact lenses), ocular wound bandage, dosage forms application for treatment of corneal wound healing such as amniotic membrane (AM) bandages, bandage contact lenses (BCL), and collagen shields (Zidan, et al., Pharm Dev Technol, 23 (3): 255-260 (2017)). Ophthalmic gels are divided into two categories: gel drops and in situ gels. The first exist as viscous solutions before application to the eye. Gel drops are simple viscous formulations that do not undergo any changes after their administration. In situ gels, by comparison, are liquids that are applied as drops onto the eye and only after administration undergo a sol-gel-to-gel transition in the conjunctival cul-de-sac following external stimuli, such as pH, temperature, or ions, with a significant improvement in ocular bioavailability The dosage form is preferably not in the form of a liquid eye drop, which is expected to be washed away by continuous tear secretion, thus necessitating more frequent reapplication. As used herein, “liquid eyedrop” refers to a liquid formulation to be applied in very small amount to the eyeball, and which remains liquid following application to the eyeball. Liquids that are applied as drops onto the eye and only after administration undergo a sol-gel-to-gel transition in the conjunctival cul-de-sac following external stimuli, such as pH, temperature, or ions, with a significant improvement in ocular bioavailability, as not included in this exclusion of “liquid eye drops”.

In a preferred embodiment, the disclosed formulations are in the form of a contact lens.

In preferred embodiments, the pharmaceutical composition containing the antimicrobial hydrogel reduces the requirement for continuous application, and reduces its removal by continuous tear secretion. Preferably, the pharmaceutical composition containing the antimicrobial hydrogel is in the form of contact lens.
1. Contact Lenses

Antimicrobial heterojunction hydrogel compositions in the form of antimicrobial hydrogel contact lenses are described. Extended wear contact lenses are usually soft contact lenses. They are typically made of flexible plastics that allow oxygen to pass through to the cornea. Fewer rigid gas permeable lenses have been designed and approved for overnight wear. Therefore, antimicrobial hydrogel contact lenses can be for daily or overnight use, or for long-term use. The antimicrobial hydrogel contact lenses can be formulated for use in therapeutic applications according to the needs of the intended recipient. For example, in some embodiments, the antimicrobial hydrogel contact lenses have the same, shape, size and contour as a contact lens, e.g., for use as a contact lens by direct application onto the surface of the eye. In some forms, the heterojunction hydrogel can be added, e.g., by coating onto commercially available contact lens. Thus, the disclosed formulations can be applied onto soft lenses made from one or more soft polymer materials including but not limited to, a hydrogel, a silicone-based hydrogel, a polyacrylamide, or a hydrophilic polymer. For example, in an aspect, contact lenses disclosed herein can include crosslinked hydrogels including hydrophilic monomers (e.g. N-Vinylpyrrolidone, N,N-dimethylacrylamide, 2-hydroxyethyl methacrylate, hydroxyethyl acrylate, methacrylic acid and acrylic acid), strengthening agents, ultraviolet light (UV) blockers, or tints. In another aspect, contact lenses disclosed herein can include silicone hydrogels (e.g. crosslinked hydrogels containing silicone macromers and monomers, as well as hydrophilic monomers that absorb water). In yet another aspect, contact lenses disclosed herein can include hard lenses made from one or more rigid materials including but not limited to, a silicone polymer, polymethyl methacrylate, or rigid gas permeable materials.

The disclosed antimicrobial hydrogel contact lenses can include one or more additional active agents. For example, the contact lenses can include one or more therapeutic, diagnostic or prophylactic agents, or a dye, or coloring agent. The disclosed antimicrobial hydrogel contact lenses can be packaged and stored in appropriate containers, such as a contact lens case. The lenses can be stored within a suitable fluid, for example, contact lens solution.
2. Ocular Wound Bandage

Ocular wound bandages including the antimicrobial heterojunction hydrogel compositions are described. Ocular wound bandages including the antimicrobial heterojunction hydrogel compositions or peptidomimetics can be formulated for use in therapeutic or cosmetic applications, according to the needs of the intended recipient. In some embodiments, the antimicrobial heterojunction hydrogel compositions are applied to commercially available ocular wound bandages, such as commercially available gauzes or eye patches. Application can occur after assembly of the antimicrobial heterojunction hydrogel materials has occurred, and can be carried out by any suitable means known in the art for application, such as spraying, coating, painting, etc.

In certain embodiments, compositions of antimicrobial heterojunction hydrogels are applied in the form of a solution or powder directly onto gauze or other non-peptide structures. For example, antimicrobial heterojunction hydrogel compositions and/or scaffold materials can be contacted with tissue around and within the eye, and held in place by a bandage or gauze, or as one component of an eye patch.

Medicated ocular bandages are reviewed in (Zidan, et al., Pharm Dev Technol, 23 (3): 255-260 (2017), discussed in brief below.
Amniotic Membrane Bandages

Amniotic membrane (AM) is an avascular fetal membrane harvested from placental tissue obtained from elective cesarean sections of pregnant women.
Bandage Contact Lenses (BCL)

A bandage lens is a therapeutic contact lens (usually soft) which can be worn eye for an extended period of time. A therapeutic bandage lens is any contact lens used to promote healing, relieve pain and protect the ocular surface. The placement of these therapeutic lenses, whether temporary or as part of a long-term treatment plan, should be thought of as a medical treatment or procedure, rather than a lens per se. It is a medical prosthesis for someone who has been injured, is necessary for the health of the eye (not for vision correction) and can minimize the risk of progressive disability, ocular morbidity or both. Commercial silicone hydrogel BCL including Biofinity®, ACUVUE®, Pure Vision®, and AIR OPTIX® are currently used after photorefractive keratectomy (PRK) to promote epithelial healing and control pain.
Polymeric Hydrogels

Hydrogels are the main components of ocular bandage lenses because of their hydrophilic nature and cross-linked polymeric networks that have high water absorbance capability. Hydroxyethyl methacrylate (HEMA) and poly (hydroxyethylmethacrylate) (p-HEMA) are currently used in soft contact lens preparation and are the most common polymeric hydrogels utilized in drug-eluting contact lenses for controlled drug release up to 1 week.
Collagen Shields

Collagen shields were first introduced in the late 80s as contact lens shaped bandages fabricated from porcine scleral tissue that resembles the collagen of the human eye now there are currently marketed collagen shields by OASIS® Medical, Inc.

### III. Methods of Use

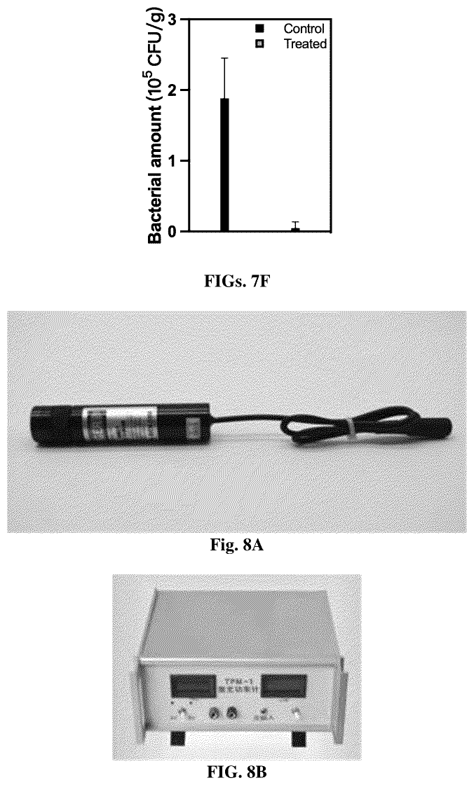

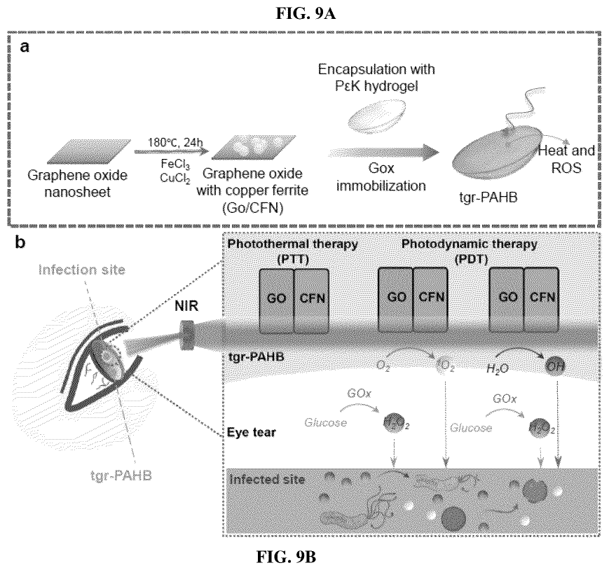

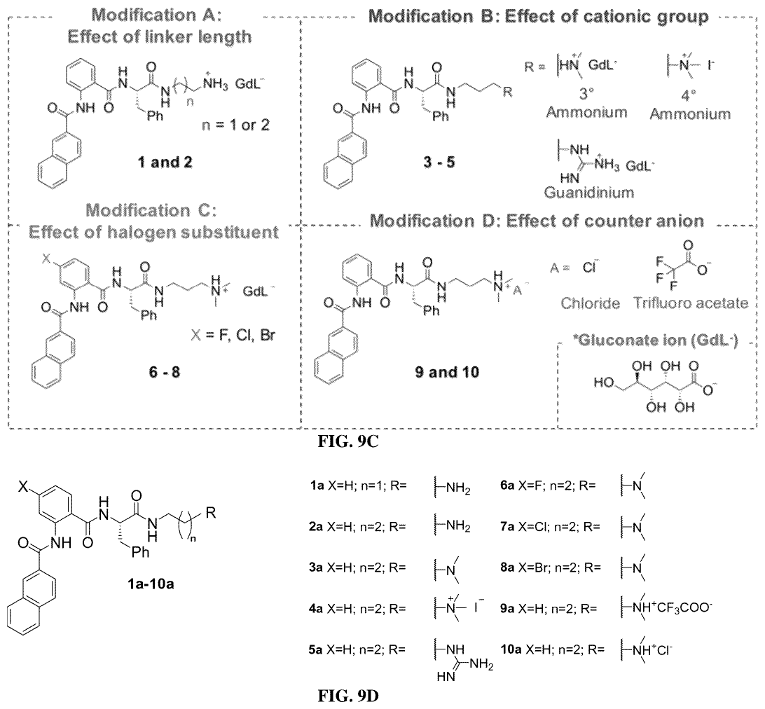

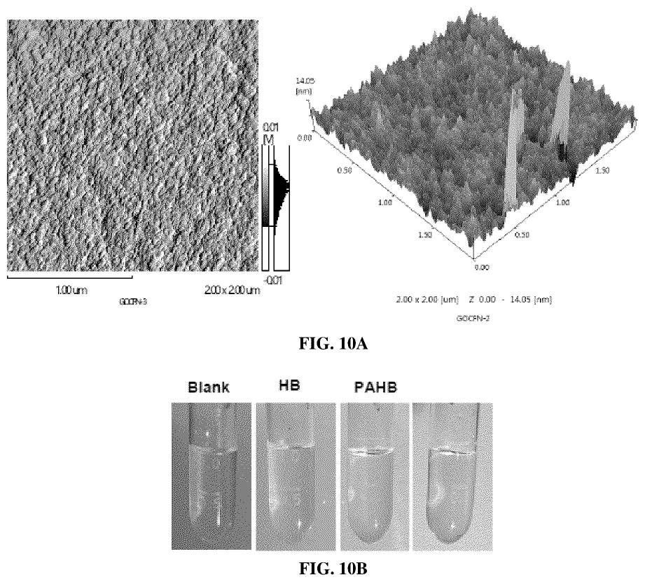

Methods for treating an ocular disease, ocular disorder or ocular condition in a subject are provided. The disclosed method includes applying a therapeutically effective amount of the disclosed antimicrobial heterojunction hydrogel compositions to a subject in need thereof, either topically or by instillation. The method includes contacting the eye of a subject in need thereof, with an effective amount of the disclosed composition. The method further includes contacting the composition in the subject's eye with near infrared radiation (NIR). In some forms, NIR is released from a portable probe, connected to a light source (with a picture). An exemplary NIR laser probe and laser source are shown in FIGS. 8A and 8B. The probe is aligned to the tgr-PAHB to deliver the treatment. In some forms, the light source is a fixed instrument in a clinic. In some forms, the light source is a portable device e.g., a handheld LED Infrared Light therapy such as Novaa Light Pro™M. In some forms, the distance during treatment is approximately from about 2 cm to about 8 cm, preferably from about 3 cm to about 5 cm, more preferably about 4 cm. In some forms, the duration of treatment is from about 20 minutes to about 30 minutes daily for about 3 days to about 5 days.

In a preferred embodiment, the subject has or has been diagnosed with microbial keratitis, and the composition is applied to the infected site.

A. Diseases/Conditions to be Treated

The disclosed antimicrobial hydrogel compositions can be used to treat a variety of conditions associated with ocular infection. For example, the antimicrobial heterojunction hydrogel compositions can be used to treat conditions of the lids including blepharitis, blepharconjunctivies, meibomianitis, acute or chronic hordeolum, chalaziori, dacryocystitis, dacryoadenities, and acne rosacea; conditions of the conjunctiva including conjunctivitis, ophthalmia neonatorum, and trachoma; conditions of the cornea including corneal ulcers, superficial and interstitial keratitis, keratoconjunctivitis, foreign bodies, and post-operative infections; and conditions of the anterior chamber and uvea including endophthalmitis, infectious uveitis.

Non-limiting examples of prophylaxis situations include treatment prior to surgical procedures such as blepharoplasty, removal of chalazia, tarsorrhapy, procedures for the canualiculi and lacrimal drainage system and other operative procedures involving the lids and lacrimal apparatus; conjunctival surgery including removal of ptyregia, pingueculae and tumors, conjunctival transplantation, traumatic lesions such as cuts, burns and abrasions, and conjunctival flaps; corneal surgery including removal of foreign bodies, keratotomy, and corneal transplants; refractive surgery including photorefractive procedures; glaucoma surgery including filtering blebs; paracentesis of the anterior chamber; iridectomy; cataract surgery; retinal surgery; and procedures involving the extra-ocular muscles. In some preferred embodiments, the patient does not suffer from dry eyes and/or has not received refractive surgery before.

The disclosed antimicrobial hydrogel bandages can be used to treat or prevent ocular infections caused by a variety of bacteria or parasites, including but not limited to one or more of the following organisms: Staphylococcus including Staphylococcus aureus and Staphylococcus epidermidis; Streptococcus including Streptococcus pneumoniae and Streptococcus pyogenes as well as Streptococci of Groups C, F, and G and Viridans group of Streptococci; Haemophilus influenza including biotype III Aegyptius); Haemophilus ducreyi; Moraxella catarrhalis; Neisseria including Neisseria gonorrhoeae and Neisseria meningitidis; Chlamydia including Chlamydia trachomatis, Chlamydia psittaci, and Chlamydia pneumoniae; Mycobacteriumincluding Mycobacterium tuberculosis and Mycobacterium avium-intracellular complex as well as atypical mycobacterium including M. marinum, M. fortuitm, and M. chelonae; Bordetella pertussis; Campylobacter jejuni; Legionella pneumophila; Bacteroides bivius; Clostridium perfringens; Peptostreptococcus species; Borrelia burgdorferi; Mycoplasma pneumoniae; Treponema pallidum; Ureaplasma urealyticum; Toxoplasma; malaria; and Nosema.

B. Routes of Administration

In some forms, a method of treating an ocular disorder includes administering to the subject an effective amount of the disclosed antimicrobial hydrogel compositions. For example, a pharmaceutical composition containing an effective amount of antimicrobial heterojunction hydrogel composition and one or more therapeutic agents can be applied to the eye of a subject in need thereof. Preferably, the composition is in a form suitable for direct application to the infected areas. In some preferred forms, the composition is in a form suitable for instillation on the cornea. In some forms, the composition is applied as a topical formulation.

In some forms, NIR is released from a portable probe, connected to a light source (with a picture), and a doctor or a patient need only align the probe to the tgr-PAHB to deliver the treatment. In some forms, the light source is a fixed instrument in a clinic. In some forms, the light source is a transportable device e.g., a handheld LED Infrared Light Therapy Pen®. In some forms, the distance during treatment is approximately from about 2 cm to about 8 cm, preferably from about 3 cm to about 5 cm, more preferably 4 cm. In some forms, the duration of treatment is from about 20 minutes to about 30 minutes daily for about 3 days to about 5 days.

C. Effective Amounts and Controls

The disclosed antimicrobial heterojunction compositions can be administered to the eye therapeutically to achieve a therapeutic benefit, or prophylactically to achieve a prophylactic benefit, or to achieve both a therapeutic and prophylactic benefit. Therapeutic benefit means treating the underlying disorder including eradication or amelioration of one or more of the symptoms associated with the underlying disorder such that the patient reports an improvement in feeling or condition, notwithstanding that the patient can still be afflicted with the underlying disorder. For example, administration of a composition to a patient suffering from a condition provides therapeutic benefit when the patient reports a decrease in the severity or duration of the symptoms associated with the condition. Therapeutic benefit also includes halting or slowing the progression of the disease, regardless of whether improvement is realized by the patient.

In preferred embodiments, the disclosed antimicrobial hydrogel bandage and/or pharmaceutical compositions thereof, are retained at the site of administration (e.g., at the surface of the cornea) for periods of time sufficient to yield a therapeutic effect. For example, when administered onto the cornea, compositions of the disclosed antimicrobial heterojunction compositions are sufficient to prevent or reduce corneal inflammation and antimicrobial keratitis.

It is to be understood that the disclosed method and formulations are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

### Examples

Antibiotics as the conventional treatment option for bacterial keratitis has raised the problems such as antimicrobial resistance and allergy, and these problems promoted innovation of alternatives for antibiotics. An exemplary antibiotics-free biocidal corneal bandage with photo-sensitive nanocomposites embedded in enzyme-modified hydrogel was designed and tested. Nanocomposites and enzyme produce reactive oxygen species (ROS) to disinfect bacteria. The nanocomposites release ROS under near-infrared light (NIR) irradiation, and the enzyme in hydrogel produces ROS by catalyzing glucose in eye tears.

Materials and Methods

The generation of ROS was characterized using colorimetric analysis, and dyes that can react with ROS were used as indicators. The antibacterial property of the bandage against Staphylococcus aureus (S. aureus) and Pseudomonas aeruginosa (P. Aeruginosa) suspended in medium was compared with a blank control group through spread plate method (N=3), Live/dead staining (N=3). The morphology of the bacteria was examined by scanning electron microscopy (SEM). The ROS level in bacteria after treated by the bandage under NIR irradiation was also evaluated with DCFH-DA.

Synthesis of Go/CFN Heterojunction

The GO/CFN heterojunctions were synthesized through hydrothermal reaction. Briefly, 0.05 g GO nanosheet (Tanfeng Tech., Suzhou, China) and 2.16 g were first dissolved in 60 mL ethylene glycol (Chengdu KeLong Reagent, China). Afterward, 2.16 g iron chloride hexahydrate (FeCl3·6H2O, Aladdin, Shanghai, China), 0.68 g copper chloride dihydrate (CuCl₂·2H₂O, Aladdin), 1.5 g sodium acetate (Aladdin), and 0.5 g polyvinylpyrrolidone (PVP, Aladdin) were added into GO-ethylene glycol solution. The mixture was ultrasonicated for 2 hours (h), and transferred to Teflon autoclave. The hydrothermal reaction was kept at 180° C. for 24 hours. Finally, the Go/CFN heterojunction powders were collected, rinsed using ethyl-alcohol and deionized (D.I.) water and dried. The same hydrothermal method was performed to synthesize CFN nanoparticles without using GO.

Preparation of Go/CFN Pεk-Gox

The procedure of Pak synthesis was similar to the antibacterial Pek hydrogel bandage which has been published.⁴⁹ Briefly, two solutions were prepared. Solution A was prepared by dissolving 0.1485 g Pεk (Bainafo Bioengineering Co. Ltd, China) and 0.0488 g Azelaic acid (Sigma) in 0.66 mL DI-water with addition of polyvinyl pyrrolidone (PVP) and 4-Methylmorpholine (NMM). Solution B was prepared by dissolving 0.05 g N-hydroxysuccinimide (Aladdin, Shanghai, China) and 0.2481 g 1-(3-Dimethylaminoproyl)-3-ethylcarbodiimide HCl (Aladdin, Shanghai, China) in 0.33 mL DI-water, and immediately transferred to ice bath. The synthesized Go/CFN and Glucose oxidase was dissolved in 5% PVP to prepare 8 mg/mL heterojunction solution and 4 mg/mL Gox solution respectively. Then, 1.23 mL of 8 mg/mL Go/CFN and 440 μL 4 mg/mL Gox were added to the mixture of solution A and B. The biocidal hydrogel bandage was obtained with polymerization overnight. The biocidal hydrogel bandage was washed by DI-water and 10% NMM to remove unreacted reagents.

Characterization of Nano-Heterojunction and Biocidal Hydrogel Bandage:

The X-ray diffractometer spectrum of heterojunction was characterized by X-ray diffractometer (XRD, Xcalibur A Ultra, Oxford, UK), adopted to detect the phase composition of samples at the Cu target radiation (λ=1.5444 Å) with a measuring range from 20° to 80°. Powders of Go, CFN and Go/CFN were dried before XRD analysis. The surface morphology and the microstructure of nano-heterojunction and biocidal hydrogel bandage were characterized using a scanning electron microscope (SEM, JSM-7500-F, JEOL, Japan) equipped with Energy dispersive spectrometry (EDS) mapping. The ξ-potentials of the Go, CFN and Go/CFN nano-heterojunction were recorded using a particle size analyzer (Zetasizer Nano, Malvern, UK). The functional groups in Go/CFN nano-heterojunction and biocidal hydrogel bandage were analyzed by an FT-IR (Nicolet 6500, Thermo Scientific, USA). The wavenumber interval for the detection was 400-4000 cm⁻¹. Water contact angle and water content are two measurements used to assess the properties of a hydrogel. The water contacts angle of the biocidal hydrogel bandage was measured by single drop technique. The water content of the biocidal hydrogel bandage was measure by the following equation:

$$\\ \begin{matrix}water content \% = \frac{{M}_{wet} - {M}_{dry}}{{M}_{dry}} \times 100 \% & Equation ( 1 )\end{matrix}$$

Where M_{wet} indicated the mass of the biocidal hydrogel bandage fully absorbed with water while M_{dry} indicated the mass of biocidal hydrogel bandage that was totally dried in oven overnight.

Water contact angle (WCA) provides a measure of wettability, hydrophobicity (if water is used), and the strength of contact of a solid surface.

The contact angle (water) was found to be less than 20 degree. Water content was found to be over 75%.

Photoelectrochemical Behavior Measurement:

The PL spectra were reported utilizing a FluoroMax-4 fluorescence spectrophotometer (Horiba, France) with an excitation wavelength of 488 nm. The photocurrent curves were obtained through a CHI660E electrochemical workstation (Chenhua, China) with a standard three-electrode system.

Photothermal Performance Assessment:

The photothermal performance of Go/CFN nano-heterojunction and biocidal hydrogel bandage was investigated by the infrared thermal imager (FLIR, E6, USA) in phosphate-buffered solution (PBS). Briefly, certain amount of nano-heterojunction or biocidal hydrogel bandage was suspended in 500 μL of PBS. The temperature of samples was recorded by a FLIR E6 IR thermal imager (USA) every 1 minute exposed under NIR exposure with varying output power. The real-time NIR thermal images were photographed by every 1 minute.

Photothermal Effect Assessment
Detection of Singlet Oxygen ¹O₂

The singlet oxygen was detected by measuring the absorbance change of 1,3-diphenylisobenzofuran (DPBF, Aladdin). DPBF ethanol solution (300 μL, 0.6 g/L) was mixed with varying concentration of GO/CFN heterojunction in darkness. A near-infrared radiation (NIR) light source was applied to the mixture of DPBF and heterojunction. The solution was diluted 10 times after treatment and the absorbance was measured by UV-vis spectrophotometer from 350 nm to 500 nm (UV-1800PC, AOELAB, Shanghai, China).²
Measurement of Hydroxyl Radical ·OH

The production of ·OH was measured by analyzing the absorbance of Methylene blue (MB, Aladdin) solution³ and 3,3,5,5-tetramethylbenzidine (TMB, Aladdin). Briefly, presence of H₂O₂ without NIR laser irradiation 40 mg/mL MB aqueous solution was incubated with heterojunction and followed by addition of 100 μL 0.5 M H₂O₂. The H₂O₂ will function as a trigger of Fenton(-like) reaction by heterojunction. Meanwhile, the NIR light source was applied, and the solution was diluted 10 times. The absorbance of the MB solution that has been treated with heterojunction was detected by a UV-vis spectrophotometer from 550 nm to 750 nm. For measurement using TMB, the samples were immersed in 500 μL mixture of TMB (0.5 g/L) and glucose (200 mg/dL). After the sample was treated, sodium acetate-acetic acid buffer (pH=4.5) was added into the mixture for color development.

Spread Plate Method:

The NIR-triggered antibacterial properties against Gram-positive Staphylococcus aureus (S. aureus, ATCC 25923) and Gram-negative Pseudomonas aeruginosa (P. aeruginosa, CMCC (B) 10 104) were assessed using the spread plate method. The biocidal hydrogel bandage was transferred into a bacterial suspension (500 μL, 10⁴ CFU/mL) containing glucose (200 mg/dL) and treated with or without NIR treatment. After the treatment, 80 μL of bacteria-containing liquid was evenly applied on LB agar plate. The S. aureus on agar plates was cultured at 37° C. for 48 hours, while the P. aeruginosa was cultured for 24 hours. The plates were photographed, and the numbers of bacteria colonies were counted. The antibacterial efficiency of the biocidal hydrogel bandage and survival rate of the bacteria were calculated as following:

$$\\ \begin{matrix}Antibacterial efficiency \% = \frac{{N}_{control} - {N}_{sample}}{{N}_{control}} \times 100 \% & ( 2 )\end{matrix}$$

$$\\ \begin{matrix}Survival rate \% = \frac{{N}_{sample}}{{N}_{control}} \times 100 \% & ( 3 )\end{matrix}$$

where N_{control} was the number of colonies on agar plate from group without treatment and was Nₛₐₘₚₗₑ the colonies from treatment groups.

Bacterial Morphology Assessment

The membrane and morphology of the bacteria were observed by SEM after varying treatment. S. aureus or P. aeruginosa, MRSA and MDR-PA (500 μL, 1×10⁶ CFU/mL) were cultured on glass coverslips (1 cm) overnight to allow bacterial attachment. 500 μL of LB broth containing glucose (200 mg/dL) was added to mantle the coverslips after removal of unattached bacteria, followed by treatment of samples under NIR or in darkness for 20 minutes. The sample treated bacteria were immobilized with glutaraldehyde solution (2.5%, v/v) for 2 hours and dehydrated in gradient ethanol (30%, 50%, 70%, 90% and 100%) for 10 minutes. The dried bacteria as well as the coverslips were coated with gold and observed under SEM (JSM-7500-F, JEOL, Japan). Bacteria were collected after treatment by centrifuge suspension at 8000 rpm for 1 minute, fixed by glutaraldehyde (2.5%, v/v) and stained by OsO₄ (1%, w/v) for 2 hours sequentially. The TEM images were obtained after staining with uranylacetate and placing the bacteria on copper grids.

Live/Dead Assay Staining

Bacteria suspension (500 μL, 10⁸ CFU/mL) was incubated in 48-well plates for 20 hours to allow bacterial attachment. The attached bacteria were treated by biocidal hydrogel bandage with NIR exposure or in darkness for 20 minutes. The treated bacteria were stained with Live/Dead BacLight bacteria viability kit (Thermo Fisher, USA) for 15 minutes under darkness. After gently washing with sterilized D.I. water, the images of stained samples were captured and evaluated with CLSM fluorescence microscope (N-SIM, Nikon, Japan). The live bacteria (green fluorescence signal) and dead bacteria (red signal) in the images were analyzed by image J.

Detection of ROS Level:

The ROS level in bacteria was measured by a ROS assay kit (DCFH-DA, Solarbio). Suspensions of S. aureus (10⁸ CFU/mL) or P. aeruginosa (10⁸ CFU/mL) were cultured in 48-well plates overnight at 37° C. to allow bacteria to attach. After removal of un-attached bacteria, 500 μL of LB broth containing glucose (200 mg/dL) was added and the bacteria were treated with samples with NIR or in darkness for 20 minutes. The DCFH-DA working solution was added and incubated with bacteria for 20 minutes after removal of the remaining solution. After few washes with sterilized D.I. water, the bacteria were imaged by an inverted fluorescence microscope (CKX53, OLYMPUS).

GSH Consumption Detection:

The capacity of the samples to deplete Glutathione (GSH, Aladdin) was measured with Ellman assay. Briefly, GSH was dissolved in 0.8 mM of carbonate buffer solution together with glucose to maintain the concentration of 200 mg/dL. After transferred with sample, the mixtures were incubated sin darkness for 30 min. The mixture of GSH and samples were treated with or without NIR after the incubation. The mixtures in darkness or with addition of H₂O₂ (1 mM, Kelong) were regarded as negative and positive controls respectively. Tris-HCl buffer (5 mM, 500 μL) and 5,5′-dithiobis-(2-nitrobenzoic acid) solution (DTNB, 10 mM, 100 μL) were added in the mixture and react for 30 minutes. The undermine of GHS was measured by the absorbance decrease of the final mixture at 410 nm.

Detection of Damage to Bacteria: ATP, DNA, Proteins, ONGP

Anti-Biofilm Test

The anti-biofilm property of samples against S. aureus and P. aeruginosa was firstly measured with crystal violet absorbed. Briefly, bacteria suspension (500 μL, 10⁸ CFU/mL) was added on glass coverslip (1 cm) and incubated for under 37° C. 5 days to allow biofilm formation with medium changing every two days. The suspended bacteria were removed, and the biofilm was washed by sterilized PBS gently before treatment. After sample treatment with NIR or in darkness for 20 minutes, the biofilm was fixed by Glutaraldehyde (2.5%, v/v) for 2 hours and then stained with crystal violet (0.5% wt, v/v) for 30 minutes.

The unstained crystal violet was removed by resining with PBS. The stained biofilm was imaged, and the amount of crystal violet trapped in biofilm was measured the absorbance at 595 nm by extracting the dye with acetic acid (33%, v/v). The biomass of biofilm was calculated as following:

$$\\ \begin{matrix}Relative biofilm biomass \% = {A}_{sample} \div {A}_{control} \times 100 \% & ( 4 )\end{matrix}$$

where Aₛₐₘₚₗₑ was the average OD value of the group without treatment while A_{control} was the absorbance of experimental groups.

Furthermore, the anti-biofilm ability was detected by stain the bacteria with Live/Dead assay (ThermoFisher, U.S.A.). The biofilm was treated with samples with NIR exposure or in darkness for 20 minutes after matured. Live/Dead assay was adopted for staining the bacteria inside of biofilm following removal of unattached bacteria. The bacteria were imaged by an inverted fluorescence microscope and the signal was analyzed with image J.

Cell Culture:

Human cornea epithelium cells (HCE-T) was used for investigation of in-vitro biocompatibility. Cells were cultured in Dulbecco's modified Eagle's medium/Nutrient Mixture F-12 (DMEM/F12), Gibco) with addition of 10% fetal bovine serum (Hyclone, USA) and 1% antibiotic-antimycotic biotics (Gibco).

Cell Viability Test:

The HCE-T cells were harvested and seeded in to 24-well plate with concentration of 2×10⁵ cell per well. Cells were incubated under 37° C. for one day before treatment to form an epithelial layer. A circular piece of filter paper which was soaked in 0.1 M of sodium hydroxide, was applied onto the central region of the epithelial layer to create an injury wound. After the injured epithelium layer was washed with PBS three times, samples were placed in the wound, and daily cultured with cells under NIR exposure or in darkness for 20 minutes. After treatment, the samples were removed, and cells were incubated with medium containing 10% of Cell Counting Kit (CCK-8, Dojindo, Japan) for 2 hours. The optical density (OD) value of supernatant at 450 nm was measured by a microplate reader. The viability of HCE-T cells was calculated as following:

$$\\ \begin{matrix}Percentage of cell viability \% = \frac{{A}_{sample} - {A}_{background}}{{A}_{blank} - {A}_{background}} \times 100 \% & ( 5 )\end{matrix}$$

Where Aₛₐₘₚₗₑ and A_{blank} were the absorbance from experimental group and untreated group respectively, A_{background} was the absorbance without cells.

Cell Scratch Experiment and Cell Migration:

The HCE-T cells were harvested and seeded in to 24-well plate with concentration of 2×10⁵ cell per well. Cells were incubated under 37° C. until the epithelial layer formed. HCE-T cells were starved with FBS-free medium one day before the experiments. A 200 μL pipette tip was employed to scratch a straight line to simulate an incised wound. Then the epithelial layer was rinsed with PBS to removed cell debris. Samples was applied to cover the straight line, and co-culture with cells under NIR or in darkness for 20 minutes. Afterwards, cells were photographed with an optical microscope (Olympus) at time points (0, 4 and 24 hours). The healing rate (8%) was calculated with following equation:

$$\\ \begin{matrix}δ \% = \frac{{A}_{0} - {A}_{t}}{{A}_{0}} \times 100 \% & ( 5 )\end{matrix}$$

where A₀ indicated the scratched are at 0 hour, and Aₜ represented the remaining scratched area at different time points.

Inducement of the Infectious on Ex Vivo Models

Fresh porcine eyes were collected from recently killed pigs. After removal of the meat, the eyes were washed with 0.9% saline and soaked with 10% (v/v) povidone iodine for 10 minutes. The epithelium layer was removed with a surgical blade after the sterilization with iodine. The cornea was excised with 2 mm of sclera surrounding and the uveal tissue was removed from the isolated cornea. Dissected cornea was soaked in PBS (1% anti-anti) for 20 minutes and followed by washing with sterilized PBS three times. The cornea was transferred to a mold and supported by 1 mL of DMEM solution with 2% (wt %) agarose (Sigma, Shanghai). 20 μL suspensions of MRSA (0.5×10⁵ CFU/mL) or MDR-PA (0.25×10⁵ CFU/mL) suspension was dropped onto the central of the cornea. The infected cornea was then incubated with antibiotics-free DMEM/F12 (5% Dextran) in incubator at 37° C. with 5% CO₂ for one day.

Treatment on Ex Vivo Models and Further Characterization:

PAHB, LVX/CAZ, tgr-PAHB were applied onto the cornea. Cornea with PAHB or tgr-PAHB was placed under the NIR irradiation with 1 W/cm² for 20 minutes. During the treatment, PBS containing 2 mg/mL glucose was dropped on the cornea at a speed of 1 μL/min to mimic the flow of eye tear. The cornea was homogenized at day 5 into 20 mL PBS and 80 μL of the homogenized solution was spread on an agar plate for counting the number of bacteria. The harvest cornea was fixed with PFA overnight and dehydrated with gradient ethanol (30%, 50%, 70%, 90% and 100%). The dried cornea was then transferred to SEM observation.

### Results and Discussion

Dyes were oxidized after treatment with bandage under NIR irradiation, and the enzymes on the bandage could also oxidize dye in darkness in the presence of glucose. The living colonies decreased significantly to (52+/−2) % (S. aureus) and (27+/−2) % (P. aeruginosa) after treated by the bandage and NIR irradiation. The antibacterial rates from Live/dead staining were ((54+/−1) % to S. aureus and (83+/−2) % to P. aeruginosa). Severe damage on bacteria membrane and ROS signals were found only in bacteria that were treated by the bandage and NIR irradiation. In summary, the tgr-PAHB strongly reduced S. Aureus and P. aeruginosa, and their drug-resistant strains, which demonstrates their applicability for the treatment of Bacterial Keratitis.

Synthesis and Characterization of GO/CFN Heterojunctions and tgr-PAHB

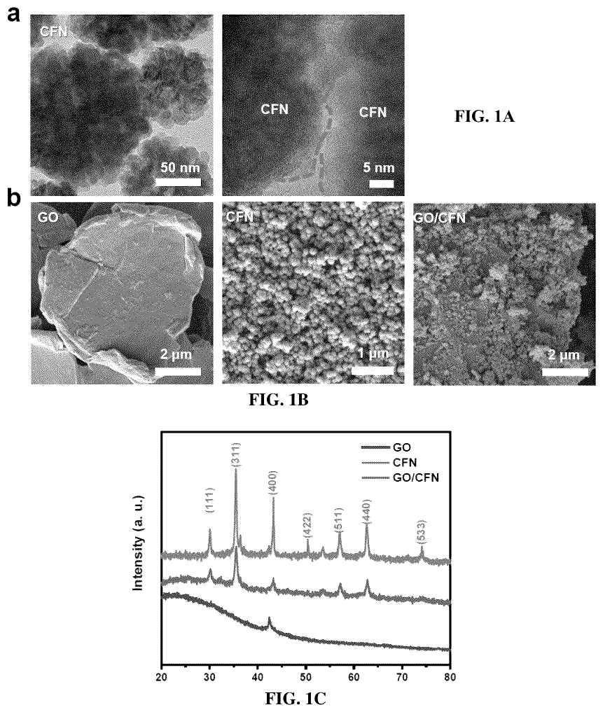

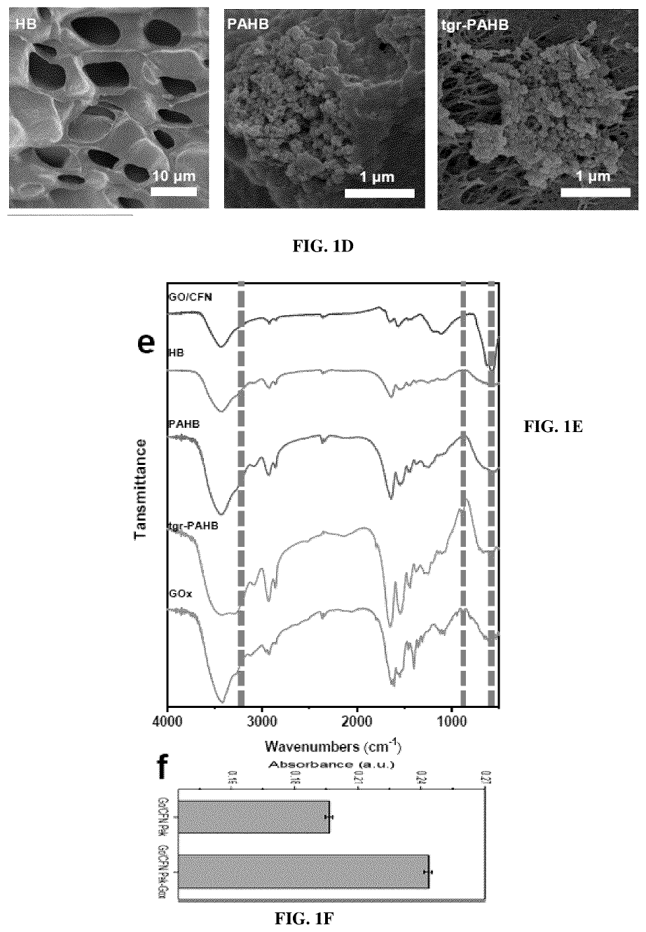

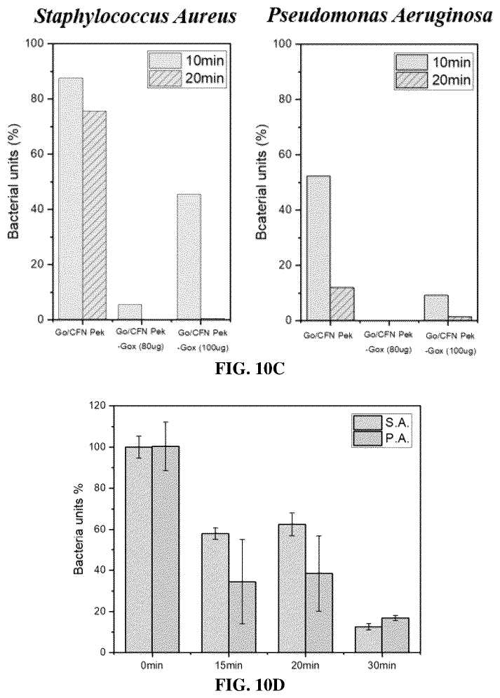

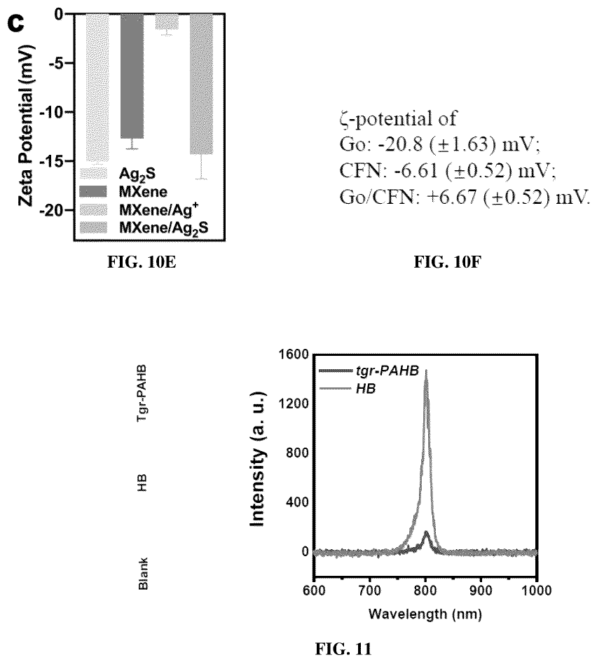

As illustrated in FIG. 9A, the encapsulated GO/CFN heterojunctions were first fabricated by in-situ growing of CFN onto GO sheets to endow photodynamic and chemodynamic property of tgr-PAHB. Large surface on GO provides abundant bonding site of CFN. The structure and morphology of CFN are determined by high-resolution TEM (HE-TEM) (FIG. 1A). The lattics spacing of cubic spinel CFN is 0.34 nm and 0.2 nm for (440) and (311) respectively. The termination groups on GO surface grant negative charge surface for GO in solution which electrostatically interacts with copper and ion irons and facilitates the deposition of CFN. This was measured by zeta-potential; the zeta-potential of GO is −20.8 mV The deposition of CFN on GO nanosheet is further confirmed through Field emission scanning electron microscope (FE-SEM) (FIG. 1B). The uniform spherical nanoparticles of CFN with the size around 120 nm, covers on the GO nanosheets. The thickness of in-situ grown CFN layer is approximately 14 nm which is consistent with atomic force microscope (AFM) (FIG. 10A). Energy dispersive spectroscopy (EDS) of GO/CFN illustrates the evenly distribution of CFN on GO. FIG. 11 shows the intensity of transmitted NIR light spot after applying hydrogel or tgr-PAHB. The phase structure of GO/CFN heterojunctions is further illustrated by XRD analysis (FIG. 1C). Typical peaks of CFN (PDF#77-0010) at 20 value of 35.49, 43.29, 57.22, 30.15 and 62.80° from curve of GO/CFN heterojunction, indicate the crystal planes of (311), (400), (511), (111) and (440).⁵⁰ After the GO/CFN heterojunctions synthesized successfully, PεK hydrogel bandage (HB) is applied to carry the GO/CFN. HB provides anchoring platform for GO/CFN heterojunctions, which prevents eye tear dilution of GO/CFN and direct physical damage by heterojunction to epithelium layer. The transparent and biocompatible PεK with high water content is considered to be an ideal material for ocular administration as a corneal bandage.^{47 48 51 52} The PεK hydrogel bandage (HB) exhibits homothetic and smooth cross-sectional morphology under FE-SEM (FIG. 1D). After encapsulating with GO/CFN heterojunction in HB, the photo-active hydrogel bandage (PAHB) is fabricated. GO/CFN heterojunctions in PAHB are distributed in the middle of cross-sectional, which indicates the GO/CFN are embedded inside instead of adhering on the surface of HB. Compared to PAHB, the immobilization with GOx further improved the hydrophilicity and porosity of tgr-PAHB. The GO/CFN heterojunctions are evenly distributed in the pores between PεEK polymer chains in tgr-PAHB (FIG. 1D). The increase of hydrophilicity is further demonstrated by measurement of the decreased contact angle and increased water content (FIG. 10F). Further investigation of the GOx chemical immobilization on tgr-PAHB is authenticated by FT-IR (FIG. 1E) and BCA protein assay (FIG. 1F). The strong absorbance at 574 cm⁻¹ indicates the presence of GO/CFN in PAHB and tgr-PAHB.⁵³ The absorbance at 1560 and 1118 cm⁻¹ corresponds to the skeletal vibration of graphene oxide (C═C) and stretching of —C—O bonds both from GOx and PεK polymers. Compared to PAHB, tgr-PAHB exhibits a peak at 3284 cm⁻¹, which indicates the symmetry vibration of amine groups (—NH₂) and the peptide proton mode of the amide band of —NHCO— from GOx.⁵⁴ Meanwhile, the amount of protein increases sharply after the bandage is modified with GOx (FIG. 1F). Furthermore, energy dispersive spectroscopy (EDS) presents homogeneous distribution of Cu, Fe, O and C in GO/CFN heterojunctions which are embedded in tgr-PAHB. The confirmation of EDS further proves the successful incorporation of GO/CFN in tgr-PAHB. As exhibited in X-ray photoelectron spectroscopy (XPS) spectrum of HB and tgr-PAHB. All signals of Fe and Cu element around 715 and 946 eV are obviously detected in tgr-PAHB, which further illustrates the successful embedding of GO/CFN into tgr-PAHB. The carbon nitrogen group (C—N), carbonyl group (C═O), and hydroxyl group (C—OH) contributes the binding energies of C1s at 285.7, 287.8, and 288.4 eV respectively. The Cu/Fe-O bonds origins from CFN in tgr-PAHB. The multivalent Cu(II)/Cu(I) and Fe(III)/Fe(II) could be observed in tgr-PAHB (data not shown), which provides the foundation for generating ·OH the Fenton(-like) reaction.

Photothermal Performance of tgr-PAHB

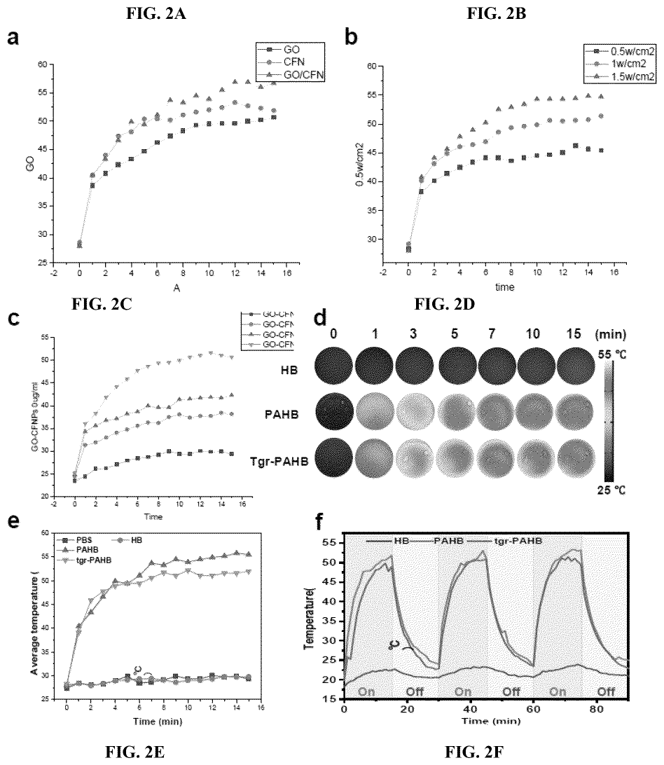

When combined with PDT, PTT eradicates pathogen in a lower temperature and strongly improves the antibacterial efficiency of PDT. To assess the photothermal property of tgr-PAHB, the photothermal curves of varying samples are obtained with NIR irradiation (808 nm). Compared to GO or CFN, the formation of GO/CFN heterojunction enhances the photothermal property in PBS solution and exhibits a higher temperature after 15 minutes irradiation (FIG. 2A). Parameters such as concentration of GO/CFN and power density of NIR can affect the photothermal performance. The impact of varying power density (0.5, 1.0, and 1.5 W/cm²) on photothermal performance to GO/CFN is investigated (FIG. 2B). Stronger photothermal effect of GO/CFN is triggered by irradiation with higher power density. Considering that pathogens can be eradicated efficiently when temperature increases above 45° C., 24 power density with 1.0 W/cm2 and is applied in further investigation. Before encapsulating GO/CFN into HB, the effects of varying GO/CFN concentration (6, 30 and 150 μg) to photothermal performance are determined in FIG. 2C. Temperature reaches 51° C. through NIR irradiation (808 nm, 1.0 W/cm²) on 150 μg of GO/CFN in PBS solution. The real-time infrared thermal images of the temperature variation further confirm the photothermal capability of tgr-PAHB under the NIR light (FIG. 2D), which is consistent with FIG. 2E. The HB exhibits negligible temperature rise after 15 min NIR irradiation, which indicates the HB lacks the ability to convert NIR irradiation into heat. After encapsulation with 150 μg GO/CFN, the PAHB and tgr-PAHB exhibits the photothermal activity between 50 to 55° C. under NIR light. The photothermal stability is measured the temperature of samples during cyclic heating-cooling. No periodic temperature changes occur in three photothermal cycles, which indicates the photothermal stability of tgr-PAHB (FIG. 2F). The bioactivity of GOx enzyme may be hindered when temperature is above 50° C.⁵⁵

Photodynamic and Chemodynamic Characterization

GOx exhibits the maximum activity between 40 to 50° C., while the sharp decrease of activity occurs when temperature increases to 55° C. These results indicate that tgr-PAHB is an excellent platform for eradicating bacteria through photothermal effect. The photocatalytic performance of a photosensitive material is related to the light absorption ability, photoexcited electron-hole separation ability and the generation of ROS. GO/CFN shows strong absorbance at NIR from 200 nm to 1000 nm.³⁷ The enhanced absorbance near 808 nm enlarges the photodynamic performance and improves the antibacterial efficiency. The photocatalytic performance can be changed by the separation and recombination of photogenerated carriers. After GO/CFN encapsulation, tgr-PAHB shows strong absorbance at 808 nm than HB (FIG. 12). Compared to GO embedding HB (HB-GO), tgr-PAHB exhibits dramatically decreased emission when excited at 345 nm wavelength. The dramatical decrease from PL spectra indicates the formation of GO/CFN heterojunction effectively suppress electron and hole recombination as the quick electron transfer from CFN to GO. As the results, the photodynamic property of GO/CFN is increased. The photocurrent responses validate the promoted separation of charge carriers and indicate the capability of producing more carriers. The results above suggest the formation of GO/CFN heterojunction improve the light absorption and carrier separation efficiency.

Production of ROS under light irradiation is a criterion for PDT. The photoactivated production of ROS by tgr-PAHB is investigated through detecting with methylene blue (MB) and 1,3-diphenyliso-benzofuran (DPBF) for hydroxyl radical (·OH)56 and singlet oxygen (1O2)57 respectively. MB captures produced OH and the absorption peak at 660 nm linear decrease with oxidation of MB by OH (FIG. 3A). HB exhibits inconspicuous changes under NIR illumination at different time points (FIG. 3B). As the results of efficiently separated electrons from holes in embedded GO/CFN, the absorbance of MB solution interacted with tgr-PAHB dramatically under NIR irradiation (FIG. 3C). Generation of large amount of ·OH is also observed under NIR exposure compared to tgr-PAHB in darkness (FIG. 3D). Similarly, reaction between DPBF and ¹O₂ and leads to the decay of absorbance at 410 nm (FIG. 3E). Negligible changes and remarkable decrease of DPBF absorbance with the presence of HB (FIG. 3F) and tgr-PAHB (FIG. 3G) respectively. Conversely, only slight decrease of DPBF absorbance occurs in tgr-PAHB group if without NIR irradiation (FIG. 3H). The mentioned results indicate that tgr-PAHB exhibits excellent photothermal effects. GO/CFN is the photosensitive agents to produce ·OH and ¹O₂ in tgr-PAHB. Furthermore, the type of produced ROS is confirmed with electron spin resonance (ESR) spectra (FIG. 3I). The signal peaks of ESR further confirm that the types of ROS generated by tgr-PAHB under NIR irradiation includes ·OH and ¹O₂.

Fenton(-like) reaction based CDT has drawn extensive attention in antibacterial strategies due to the fast cascade reaction. The presence of redox pairs of Fe(III)/Fe(II) and Cu(II)/Cu(I) in GO/CFN ensure the generation ·OH in CDT. Chromogenic reaction of 3,3′,5,5′-tetramethy benzidine (TMB) is applied to monitor the chemodynamic performance of tgr-PAHB. Colorless TMB can be oxidized by ·OH into colored oxTMB under acidic conditions (FIG. 3I). To avoid the effect of photodynamic property, the investigation is conducted in darkness with the presence of H₂O₂ (FIG. 3J). Brighter green color is observed in PAHB and tgr-PAHB group, while no oxidation occurs in HB. The strong absorbance at 655 nm occurs GO/CFN in PAHB or tgr-PAHB indicates the achievement of CDT through Fenton(-like) reaction by catalyzing H2O2 into ·OH. Furthermore, the enhancement of CDT by GOx is illustrated in darkness with the presence of glucose (FIG. 3K). The oxidation of TMB only occurs in tgr-PAHB group rather than rest of groups, which indicates H2O2 is produced by GOx and catalyzed by GO/CFN through Fenton(-like) reaction. These results suggest that the CDT of tgr-PAHB starts from catalyzing glucose surrounding environment and ends at the production of ·OH through Fenton(-like) reaction.

Glutathione (GSH) plays a dominating role in antioxidant defense which protect bacterial from damage by ROS. The antibacterial tgr-PAHB exhibits excellent consumption of GSH owing to the outstanding ROS generation ability under irradiation. The depletion of GSH substantially destroy the antioxidant defense system of bacteria. The GSH consumption ability of tgr-PAHB is evaluated through Ellman's assay (FIG. 13B). With the irradiation of NIR, tgr-PAHB consumes nearly all GSH under NIR illumination compared to HB. High depletion of GSH by tgr-PAHB in darkness occurs with the contribution of CDT. Massive ROS can be generated by tgr-PAHB under NIR irradiation as the result of GO/CFN and GOx, which suggests the tgr-PAHB is a potential strategy in treatment of BK.In vitro antibacterial activity

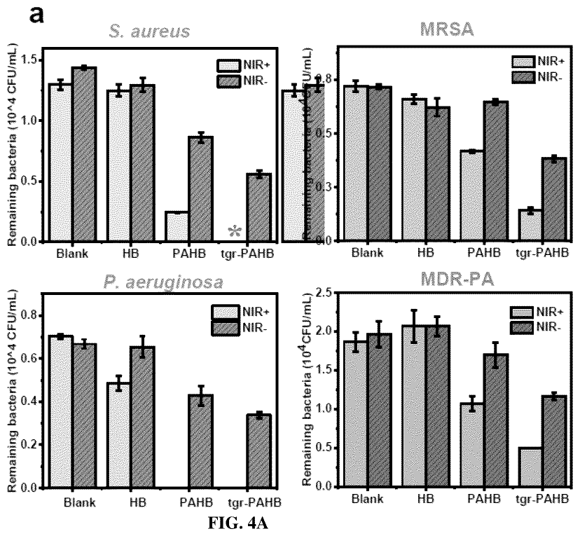

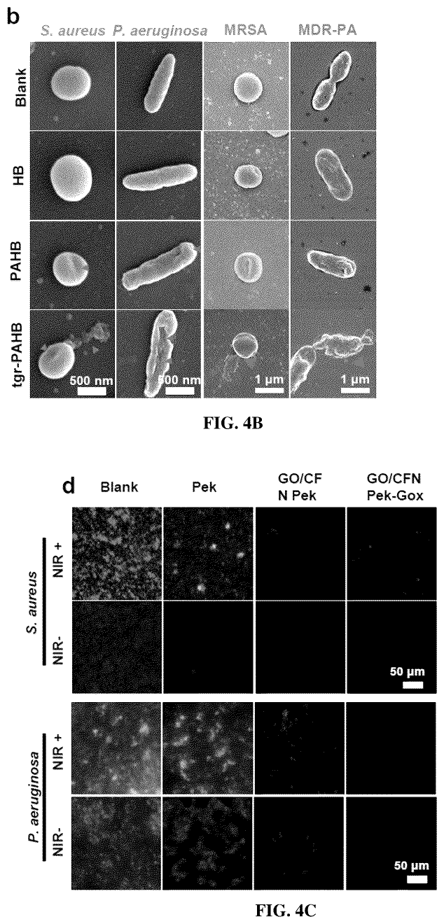

Encouraged by the excellent photothermal, photodynamic and chemodynamic performance of tgr-PAHB. The disinfection property of this corneal bandage is investigated with four strains of bacteria in vitro, including Gram-positive S. aureus and Gram-negative P. aeruginosa, as well as their drug-resistant strain MRSA and MDR-PA in vitro (FIG. 4A). The antibacterial efficiency of corneal bandages with NIR illumination (808 nm, 1 W/cm², 20 min) or in darkness is illustrated in FIG. 4A and FIG. 4B through spread plate method. To mimic the infectious micro-environment of ocular surface, addition of glucose is applied to bacterial solution as the content of eye tear. Compared to blank control group, HB exhibits a negligible eradication against the both non-resistant and drug-resistant strains with or without NIR exposure. In contrast, both PAHB and tgr-PAHB exhibit adorable antibacterial efficiency under NIR irradiation. With the encapsulation of GO/CFN, the remaining bacteria decreases to 17.0±0.2% (S. aureus), 0±0% (P. aeruginosa), 58.1±0.8% (MRSA) and 54.2±4.7% (MDR-PA) after treated with PAHB under NIR. The eradication by PAHB indicates the GO/CFN can inhibit bacteria through PDT and PTT. The immobilization of GOx further decreases the remaining bacteria to 0±0% (S. aureus), 0±0% (P. aeruginosa), 19.7±2.1% (MRSA) and 25.4±0% (MDR-PA) after treated with tgr-PAHB under NIR. The enhanced suppress of bacterial viability to 39.0±2.1% (S. aureus), 50.5±2.2% (P. aeruginosa), 53.5±2.2% (MRSA) and 59.3±2.4% (MDR-PA) by tgr-PAHB comparing to PAHB in darkness, suggests the catalysis of glucose further improved the chemodynamic performance. Remarkable bacterial viability decreases after treatment with tgr-PAHB under NIR irradiation or in darkness indicates the tgr-PAHB has the potential in BK therapy because of the eradication through synergistic strategy of CDT/PDT/PTT.

Apart from the spread plate method, the antibacterial efficiency against the four strains of bacteria was also evaluated by LIVE/DEAD staining assay under NIR (data not shown) to further confirm the eradiation against pathogens. Live bacteria present green fluorescence signal, while dead bacteria exhibit red signals in the dark filed microscopy images. Similar to blank control group, bacteria treated with HB display obvious green fluorescence with or without NIR exposure. The red signals augment after the bacteria are treated with tgr-PAHB without NIR exposure. Moreover, 43% (S. aureus), 82% (P. aeruginosa) dead bacteria are observed in the PAHB group. 55% (S. aureus), 87% (P. aeruginosa), 72% (MRSA) and 95% (MDR-PA) dead bacteria are observed in tgr-PAHB group. Notably, tgr-PAHB group presented large amount of red fluorescence, which indicates an excellent antibacterial effect toward four strains of bacteria. Subsequently the morphology of bacteria is further investigated to explore deeper the damage to bacteria caused by tgr-PAHB. SEM observation is applied to the four strains of bacteria. After treated with samples under NIR illumination (FIG. 4C) or in darkness (FIG. 12), obvious morphological and structural alterations of S. aureus, P. aeruginosa, MRSA and MDR-PA are observed in SEM images. The bacteria exhibit complete spherical or rod morphology after treated by HB with or without NIR exposure. In PAHB groups, partial shrinkage of the bacterial membrane is observed on bacteria with NIR exposure, while barely any morphology changes in darkness. In contrast, membrane deformation is observed in Pseudomonas treated by tgr-PAHB in darkness, which suggests the antibacterial effect caused by CDT and catalysis of glucose. More importantly, severe membrane leakage and shrinkage occurs on Staphylococci and Pseudomonas respectively after treated with tgr-PAHB and NIR irradiation, which implying the remarkable antibacterial effect caused by synergistic effect of CDT/PDT/PTT. The severe damage to bacterial membrane is observed after treatment with tgr-PAHB under NIR, the permeability of bacterial membrane, leakage of DNA and proteins arc investigated. By treating with tgr-PAHB, the permeability sharply increased. Meanwhile, large amount of leaked bacterial DNA and protein are detected after treated with tgr-PAHB with NIR irradiation. Furthermore, the amount of ROS inside of bacteria is investigated by ROS assay (FIG. 4E). The ROS inside of bacteria is stained in green and observed under fluorescence microscope. The bacteria treated by tgr-PAHB presents higher ROS level than PAHB, while HB barely had no ROS signals in bacteria. The bacteria also exhibit slight fluorescence signal after treated by tgr-PAHB in darkness. These results indicate that ROS damage the bacterial membrane and causes intracellular oxidative stress directly at cytoplasm in bacteria.

In Vitro Antibiofilm Activity

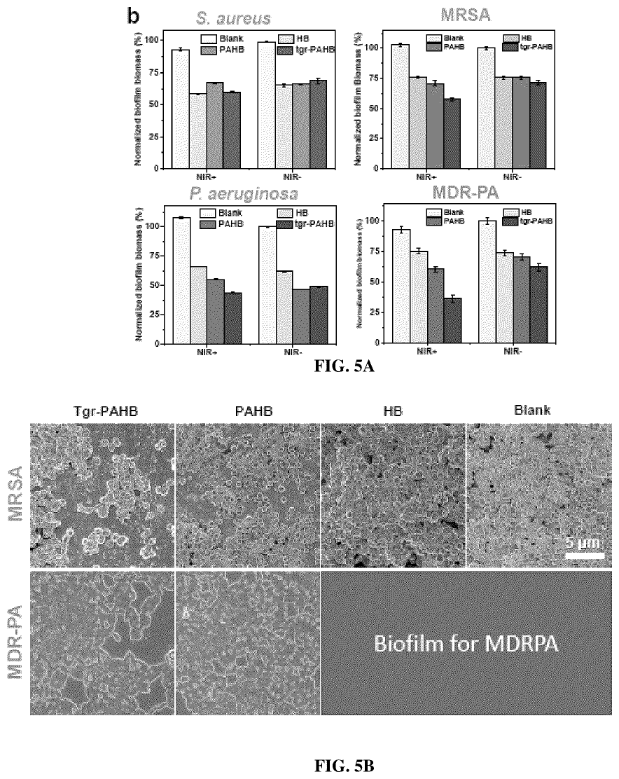

The formation of biofilm in severe BK cases hinders the treatment efficacy of antibacterial drugs and delays the healing process of the wound in epithelium layer. Mature biofilms are more resistant to conventional antibiotics and protect the bacteria from eradicated from administrated antibacterial reagents. As the result, inhibiting the formation of biofilm is an important aspect in BK therapy. ROS as one of the broad-spectrum antibacterial agents, show effective damage to biofilm.⁵⁸ Therefore, it is urgent to investigate the intrinsic anti-biofilm effect of tgr-PAHB. Mature biofilms of S. aureus, P. aeruginosa, MRSA and MDR-PA were treated with different corneal bandage with or without NIR light (808 nm, 1 W/cm2, 20 min). After that, the residual mass of biofilms and bacterial viability in biofilm are detected by crystal violet (CV) staining and LIVE/DEAD staining respectively. As shown in FIG. 5A, the addition of HB exhibits strong antibiofilm activity, which may contributed by the intrinsic antibiofilm property of PεK in HB.59 GO/CFN in PAHB reduces the remaining biofilm mass (24.7˜42%) through antibacterial strategy of PDT/PTT comparing to HB under NIR exposure. With NIR irradiation, tgr-PAHB strongly removes the biofilm. By enhanced with GOx, 39.5±0.5% (S. aurcus), 55.8±0.3% (P. acruginosa), 42.4±1.8% (MRSA) and 63.8±1.2% (MDR-PA) of the biofilm are eliminated by tgr-PAHB, which is the strongest eradication towards biofilm among all the groups. Apart from the removal of biofilm mass, the biocidal effect against bacteria in the biofilm is also important during the BK therapy. Confocal 3D images of LIVE/DEAD staining (data not shown) are adopted to investigate the bacterial viability in biofilm after treated with corneal bandages. Compared to the blank control group, the bacteria treated with tgr-PAHB induces more than half of the bacterial death to the four stains. The vast cradication of bacteria in biofilm, indicates that the tgr-PAHB can efficiently kill bacteria through penetrating ROS into biofilm. The above findings all suggest that the tgr-PAHB can effectively eradicates the mature biofilms and kill the bacteria in biofilm through synergistic therapy of CDT/PDT/PTT.

In Vitro Biocompatibility

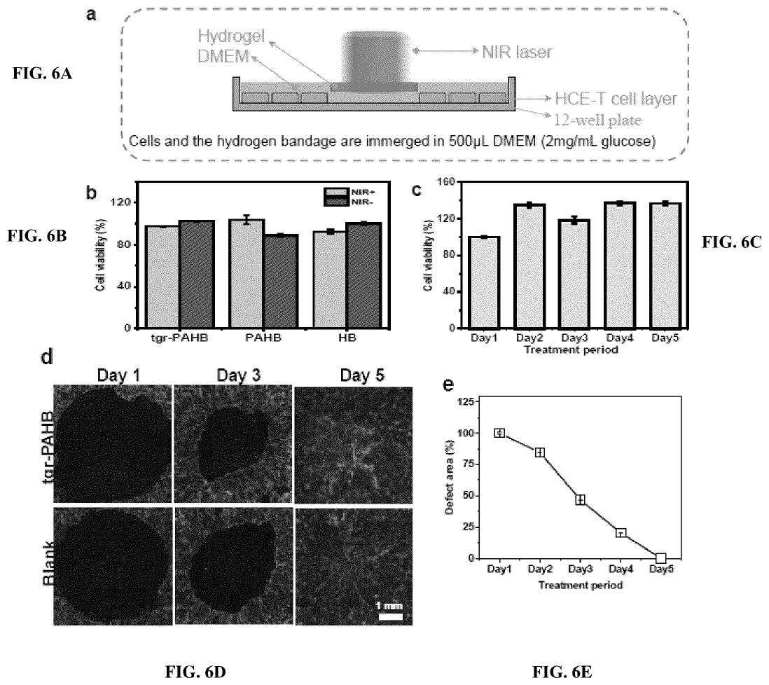

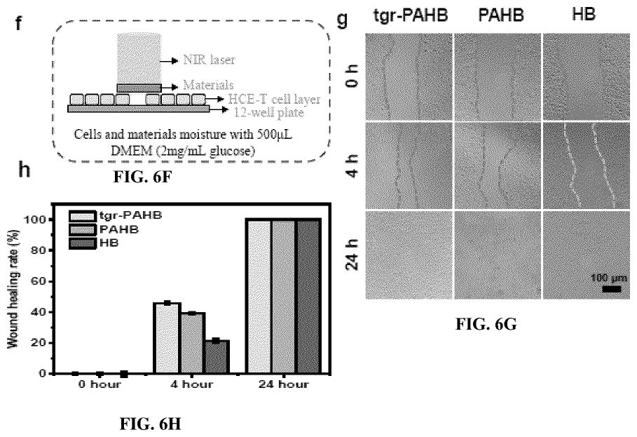

Considering that the generated heat and ROS, as well as the released copper ions from tgr-PAHB is a broad-spectrum reagent which has the potential in harming cells. The cell viability is conducted with human corneal epithelial cells (HCE-T) and measured by Cell Counting Kit (CCK-8). To explore the biocompatibility of tgr-PAHB towards the surrounding healthy tissue, an injury is induced in the central of the HCE-T layer by filter paper soaked with sodium hydroxide which mimics the wound in infected ocular surface. Different corneal bandage is cut into the same size with the filter paper and placed directly on the epithelial injury (FIG. 6A). As illustrated in FIG. 6b, both tgr-PAHB and PAHB exhibit excellent biocompatibility with or without NIR exposure (808 nm, 1 W/cm², 20 minutes), in which more than 90% of the HCE-T are alive after treated by different corneal bandages. The viability results of tgr-PAHB indicates the generated heat and ROS are not efficient in expanding to and damage the surrounding healthy epithelium.

Inspired by daily administration of eye drops in clinical BK therapy, the biocompatibility in daily treatment is conducted by measuring the viability of injured epithelial layer treated by tgr-PAHB and NIR (808 nm, 1 W/cm², 20 minutes). Cells gradually proliferate the recover during the daily treatment (FIG. 6C). Furthermore, the defect area of the injury is imaged and analyzed after daily treated by tgr-PAHB (FIGS. 6D-E). The defect area decreases gradually as the HCE-T proliferate and fill the injured area during several treatments. Meanwhile, the migration of epithelial cells after treated by tgr-PAHB and NIR (808 nm, 1 W/cm², 20 minutes) is also investigated through cell scratch experiment. Scratched lines are induced in epithelial layer and the is co-cultured with the HCE-T cells (FIG. 6F). After treated with corneal bandage, HCE-T cells at the scratched line gradually migrate in (FIG. 6G). PAHB improves the migration of HCE-T Compared to HB, while tgr-PAHB exhibits the highest migration rate (FIG. 6H). The results indicate that the wound healing process is not hindered by treatment with tgr-PAHB and NIR.

All these results indicate that the tgr-PAHB is biocompatible under NIR exposure during treatment. The healthy cells around the injury received minor damage by hyperthermia and ROS by tgr-PAHB, which suggests the tgr-PAHB has the potential in cradicating pathogens without damage the surrounding healthy epithelium.

Antibacterial Efficacy on Ex-Vivo Model

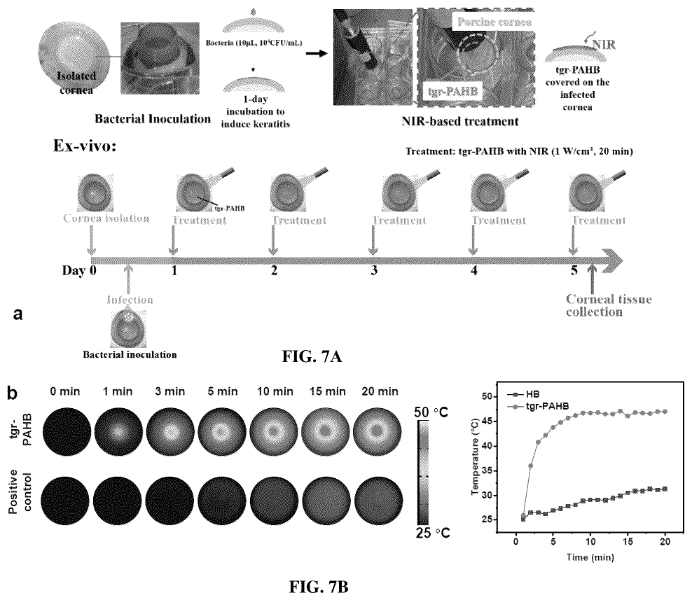

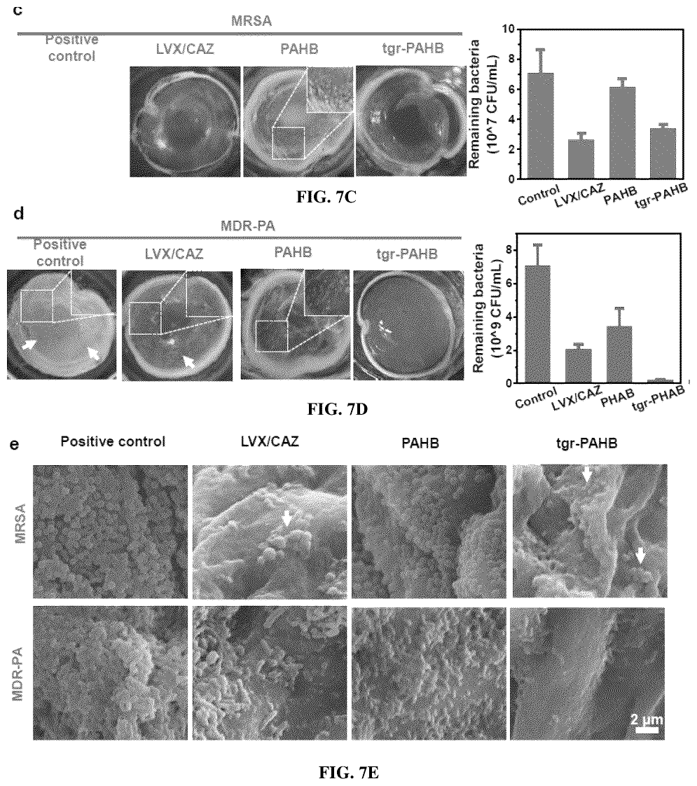

Fostered by the excellent antibacterial property towards Staphylococci and Pseudomonas in vitro, the treatment efficacy is further investigated on AMR bacteria of MRSA or MDR-PA induced BK. Due to similarity with human on the size and structure, pig eyes are usually studied as ex-vivo model.⁶⁰ To evaluated the treatment efficacy of tgr-PAHB, clinically applied fortified antibiotics levofloxacin (LVX) and ceftazidime (CAZ) are combined as negative control. LVX as a fluoroquinolone has strong biocidal ability against Pseudomonas, while CAZ as the cephalosporin shows outstanding eradication towards Staphylococci. The establishment of infected ex-vivo model and daily treatment with tgr-PAHB and NIR irradiation on porcine eyes is shown in FIG. 7A. During the treatment, PBS containing glucose is applied to mimic the infectious environment on ocular surface. After applying NIR to tgr-PAHBT on porcine cornea, the temperature sharply increases and maintains at 47° C., while the HB has no photothermal effect (FIG. 7B). FIG. 7C and FIG. 7D presents images of the porcine cornea at day 1 day day 5, induced by MRSA and MDR-PA respectively.

One of the symptoms from Staphylococci-induced BK in clinical cases is white colonies on ocular surface. In FIG. 7B, the infection at beginning is not obvious, and the colonies form at day 5. The number of bacterial colonies significantly decrease after treated with tgr-PAHB compared to positive control group. The cornea swells (bight grey area) with absence of tgr-PAHB treatment. The cornea treated with tgr-PAHB exhibits less MRSA growth at day 5 (FIG. 7E). Half of the MRSA was eradicated by tgr-PAHB (log reduction: 0.3) which is more efficient than LVX/CAZ (log reduction: 0.1) treatment.

Differ from the cytotoxicity to corneal cells contributed by Staphylococci, Pseudomonas caused damages relates to the degradation of collagen in stroma. FIG. 7D exhibits the images of infected cornea treated with varying method. In the beginning of infection, a thin biofilm of MDR-PA is formed on porcine cornea. After treated with tgr-PAHB, the formation of biofilm was inhibited and the cornea maintains transparency at day 5, while the biofilm still can be observed in PAHB, and LVX/CAZ treated group. Furthermore, the degradation of collagen in cornea is obvious in positive control group which turns the edema and non-transparency on cornea. Apart from the inhibition of biofilm and edema, tgr-PAHB suppresses the growth of MDR-PA on cornea at day 5 (FIG. 7E). The bacteria in cornea that treated by tgr-PAHB (log reduction: 2) significantly decreases compared to cornea from positive control group and LVX/CAZ (log reduction: 0.6) treated group. These results proved that tgr-PAHB has extraordinary treatment potential against MDR-PA induced BK. The SEM images further illustrates the cradication of MDR-PA on porcine cornea (data not shown). FIG. 7F shows isolated bacteria from the collected tissue from rabbit eyes. Control=the positive control group; treated=the treatment group. OD of the bacterial culture medium with collected tissue cultured in for 15 hours. The OD of treatment group was <0.1, compared to an OD>0.5 of the positive control group. MDR-PA can form a thick biofilm on porcine cornea if without any treatment, while the eradication by tgr-PAHB inhibits the growth of bacteria and formation of biofilm.

Conclusions

The catalysis of the dyes indicated that the biocidal corneal bandage produces adequate ROS in the presence of glucose with NIR irradiation. The decrease in living colonies following treatment with the bandage, indicates that the bandage has excellent antibacterial property. The in-vitro results suggested that the bandage has the potential to treat bacterial keratitis.

A tear glucose-responsive and photoactive antimicrobial hydrogel bandage (tgr-PAHB) was synthesized for rapid treatment of bacteria-induced keratitis. Compared to the traditional solutions such as broad-spectrum antibiotics in clinical application, tgr-PAHB can disinfect drug-resistant bacteria without leading of AMR. The encapsulation of photosensitive GO/CFN heterojunction, allows the tgr-PAHB eliminate bacterial through the synergistic antibacterial strategy of CDT/PDT/PTT. The utilization of NIR allows tgr-PAHB eradicate bacteria non-invasively and produce less photodamage to cornea tissue. Specifically, by immobilization with Gox, tgr-PAHB can response to eye tear and effectively consume the tear glucose hence suppress the growth of bacterial pathogens. By oxidase of tear glucose, GO/CFN further catalyze the produced H₂O₂ to produce a considerable amount of ·OH. The tgr-PAHB shows rapid and severe cradication towards Staphylococci and Pseudomonas and their drug-resistant strains through bacterial membrane damage and improve the intracellular ROS level. Furthermore, tgr-PAHB indicates superior eradication to biofilm, which provides the possibility in treating severe ocular infection. Apart from the excellent antibacterial performance, the tgr-PAHB also exhibits biocompatibility toward HCE-T cells and promotes the healing of injury on HCE-T cell layer in vitro. The treatment efficacy of tgr-PAHB against BK which is induced by drug-resistant Staphylococci or Pseudomonas, is investigated both on ex-vivo porcine cornea model and in vivo rat models. The tgr-PAHB shows more rapid and strong eradication and well efficacy within 7 days compared to clinical applied fortified antibiotics (LVX/CAZ). Meanwhile, the treatment of tgr-PAHB indicates no damage to structure of corneal epithelium and collage fibers. Accordingly, this work contributes to the design of infectious environment-responsive corneal bandage that effectively treats pathogen-induced BK.

### REFERENCES

1. Khor, et al. The Asia cornea Society infectious keratitis study: a prospective multicenter study of infectious keratitis in Asia. 2018; 195:161-70.
2. Gopinathan, et al. Review of epidemiological features, microbiological diagnosis and treatment outcome of microbial keratitis: experience of over a decade. 2009;57 (4): 273.
3. Jeng B, McLeod SJBJOO. Microbial keratitis: BMJ Publishing Group Ltd, 2003:805-06.
4. Ting, et al. Infectious keratitis: an update on epidemiology, causative microorganisms, risk factors, and antimicrobial resistance. 2021;35 (4): 1084-101.
5. Wang, et al. Prevalence and causes of corneal blindness. Clin Exp Ophthalmol 2014;42 (3): 249-53. doi: 10.1111/ceo.12164 [published Online First: 2013 Jul. 13]
6. Urwin, et al. Corneal infection models: tools to investigate the role of biofilms in bacterial keratitis. 2020;9(11): 2450.
7. Schaefer, et al. Bacterial keratitis: a prospective clinical and microbiological study. 2001;85 (7): 842-47.
8. Callegan, et al. Corneal virulence of Staphylococcus aureus: roles of alpha-toxin and protein A in pathogenesis. 1994;62 (6): 2478-82.
9. Gupta N, Mukhija R, Tandon R. Corneal Infection and Inflammation: A Colour Atlas: CRC Press 2021.
10. Breidenstein E B, de la Fuente-Núñez C, Hancock REJTim. Pseudomonas aeruginosa: all roads lead to resistance. 2011; 19 (8): 419-26.
11. Lin, et al. Genus Distribution of Bacteria and Fungi Associated with Keratitis in a Large Eye Center Located in Southern China. Ophthalmic Epidemiol 2017;24 (2): 90-96. doi: 10.1080/09286586.2016.1254250 [published Online First: 2016 Dec. 15]
12. Asbell, et al. Trends in Antibiotic Resistance Among Ocular Microorganisms in the United States From 2009 to 2018. JAMA Ophthalmol 2020;138(5):439-50. doi: 10.1001/jamaophthalmol.2020.0155 [published Online First: 2020 Apr. 10]
13. Yu, et al. A Targeted Photosensitizer Mediated by Visible Light for Efficient Therapy of Bacterial Keratitis. Biomacromolecules 2021 doi: 10.1021/acs.biomac.1c00461 [published Online First: 2021 Aug. 13]
14. Murray P R, Rosenthal K S, Pfaller M A. Medical microbiology E-book: Elsevier Health Sciences 2020.
15. O'Donnell, et al. Dose, duration, and animal sex predict vancomycin-associated acute kidney injury in preclinical studies. 2018;51 (2): 239-43.
16. Hamblin M R. Antimicrobial photodynamic inactivation: a bright new technique to kill resistant microbes. Curr Opin Microbiol 2016;33:67-73. doi: 10.1016/j.mib.2016.06.008 [published Online First: 2016 Oct. 19]
17. Kashef, et al., Can microbial cells develop resistance to oxidative stress in antimicrobial photodynamic inactivation? Drug Resist Updat 2017;31:31-42. doi: 10.1016/j.drup.2017.07.003 [published Online First: 2017 Sep. 5]
18. Bourke, et al. Microbiological interactions with cold plasma. J Appl Microbiol 2017;123 (2): 308-24. doi: 10.1111/jam.13429 [published Online First: 2017 Mar. 1]
19. Zhou, et al. Recent Progress in Photocatalytic Antibacterial. ACS Appl Bio Mater 2021;4(5): 3909-36. doi: 10.1021/acsabm.0c01335 [published Online First: 2022 Jan. 11]
20. Kremer M J P C C P. Mechanism of the Fenton reaction. Evidence for a new intermediate. 1999;1 (15): 3595-605.
21. Liu, et al. Multifunctional Magnetic Copper Ferrite Nanoparticles as Fenton-like Reaction and Near-Infrared Photothermal Agents for Synergetic Antibacterial Therapy. ACS Appl Mater Interfaces 2019; 11 (35): 31649-60. doi: 10.1021/acsami.9b10096 [published Online First: 2019 Aug. 14]
22. Fonda-Pascual, et al. In situ production of ROS in the skin by photodynamic therapy as a powerful tool in clinical dermatology. 2016;109:190-202.
23. Perez-Laguna, et. al. A combination of photodynamic therapy and antimicrobial compounds to treat skin and mucosal infections: a systematic review. 2019; 18 (5): 1020-29.
24. Zhang, et al. Synergistic antibacterial activity of physical-chemical multi-mechanism by TiO2 nanorod arrays for safe biofilm eradication on implant. 2021;6 (1): 12-25.
25. Khan, et al. Recent progress and strategies to develop antimicrobial contact lenses and lens cases for different types of microbial keratitis. Acta Biomater 2020; 113:101-18. doi: 10.1016/j.actbio.2020.06.039 [published Online First: 2020 Jul. 6]
26. Makdoumi K, Backman A. Photodynamic UVA-riboflavin bacterial elimination in antibiotic-resistant bacteria. Clin Exp Ophthalmol 2016;44 (7): 582-86. doi: 10.1111/ceo.12723 [published Online First: 2016 Oct. 21]
27. Kessel, et al. Photodynamic inactivation of pathogens causing infectious keratitis. Optical Methods for Tumor Treatment and Detection: Mechanisms and Techniques in Photodynamic Therapy XXIII, 2014.
28. Zhu, et al. Bacteria-Targeting Photodynamic Nanoassemblies for Efficient Treatment of Multidrug-Resistant Biofilm Infected Keratitis. Advanced Functional Materials 2021 doi: 10.1002/adfm.202111066
29. Martins, et al. Antimicrobial efficacy of riboflavin/UVA combination (365 nm) in vitro for bacterial and fungal isolates: a potential new treatment for infectious keratitis. Invest

Ophthalmol Vis Sci 2008;49 (8): 3402-8. doi: 10.1167/iovs.07-1592 [published Online First: 2008 Apr. 15]
30. Hunter, et al. The susceptibility of the retina to photochemical damage from visible light. 2012;31 (1): 28-42.
31. Gorgels, et al., Science v. Ultraviolet and green light cause different types of damage in rat retina. 1995;36 (5): 851-63.
32. RINGVOLD AJAO. Damage of the cornea epithelium caused by ultraviolet radiation: a scanning electron microscopic study in rabbit. 1983;61 (5): 898-907.
33. Pan, et al. Heterojunction Nanomedicine. Adv Sci (Weinh) 2022;9 (11): e2105747. doi: 10.1002/advs.202105747 [published Online First: 2022 Feb. 18]
34. Liu Y, Tian Y, Han Q, et al. Synergism of 2D/1D MXene/cobalt nanowire heterojunctions for boosted photo-activated antibacterial application. Chemical Engineering Journal 2021;410 doi: 10.1016/j.cej.2020.128209
35. Wang, et al. Magnetic ordered mesoporous copper ferrite as a heterogeneous Fenton catalyst for the degradation of imidacloprid. Applied Catalysis B: Environmental 2014;147:534-45. doi: 10.1016/j.apcatb.2013.09.017
36. Malana, et al., Adsorption studies of arsenic on nano aluminium doped manganese copper ferrite polymer (MA, VA, AA) composite: Kinetics and mechanism. 2011;172 (2-3): 721-27.
37. Zhang, et al. Copper ferrite heterojunction coatings empower polyetheretherketone implant with multi-modal bactericidal functions and boosted osteogenicity through synergistic photo/Fenton-therapy. Chemical Engineering Journal 2021;422 doi: 10.1016/j.cej.2021.130094
38. Qiao, et al. Light-Activatable Synergistic Therapy of Drug-Resistant Bacteria-Infected Cutaneous Chronic Wounds and Nonhealing Keratitis by Cupriferous Hollow Nanoshells. ACS Nano 2020; 14 (3): 3299-315. doi: 10.1021/acsnano.9b08930 [published Online First: 2020 Feb. 13]
39. Tiffany. The normal tear film. 2008;41:1-20.
40. King-Smith, et al. The thickness of the human precorneal tear film: evidence from reflection spectra. 2000;41 (11): 3348-59.
41. FRIEDRICH, et al. Theoretical corneal permeation model for ionizable drugs. 1993;9 (3): 229-49.
42. Palakuru, et al. Effect of blinking on tear volume after instillation of midviscosity artificial tears. 2008; 146 (6): 920-24.
43. Jünemann A G, Chora̧giewicz T, Ozimek M, et al. Drug bioavailability from topically applied ocular drops. Does drop size matter? 2016;1 (1): 29-35.
44. Parisi, et al. Smart bandage based on molecularly imprinted polymers (Mips) for diclofenac controlled release. 2018; 11 (4): 92.
45. Ross A E, Bengani L C, Tulsan R, et al. Topical sustained drug delivery to the retina with a drug-eluting contact lens. 2019;217:119285.
46. Hyldgaard, et al. The antimicrobial mechanism of action of epsilon-poly-l-lysine. 2014;80 (24): 7758-70.
47. Gallagher, et al. A Novel Peptide Hydrogel for an Antimicrobial Bandage Contact Lens. Adv Healthc Mater 2016;5 (16): 2013-8. doi: 10.1002/adhm.201600258 [published Online First: 2016 Jun. 9]
48. Gallagher, et al. Development of a Poly-epsilon-Lysine Contact Lens as a Drug Delivery Device for the Treatment of Fungal Keratitis. Invest Ophthalmol Vis Sci 2017;58 (11): 4499-505. doi: 10.1167/iovs.17-22301 [published Online First: 2017 Sep. 6]
49. Aveyard, et al. Antimicrobial Nitric Oxide Releasing Contact Lens Gels for the Treatment of Microbial Keratitis. ACS Appl Mater Interfaces 2019;11 (41): 37491-501. doi: 10.1021/acsami.9b13958 [published Online First: 2019 Sep. 19]
50. Zhang, et al. One-step facile solvothermal synthesis of copper ferrite-graphene composite as a high-performance supercapacitor material. ACS Appl Mater Interfaces 2015;7 (4): 2404-14. doi: 10.1021/am507014w [published Online First: 2015 Jan. 15]
51. Lace, et al. Characterization of Tunable Poly-epsilon-Lysine-Based Hydrogels for Corneal Tissue Engineering. Macromol Biosci 2021;21 (7): e2100036. doi: 10.1002/mabi.202100036 [published Online First: 2021 May 7]
52. Kennedy, et al. Antimicrobial Activity of Poly-epsilon-lysine Peptide Hydrogels Against Pseudomonas aeruginosa. Invest Ophthalmol Vis Sci 2020;61 (10): 18. doi: 10.1167/iovs.61.10.18 [published Online First: 2020 Aug. 11]
53. Kumar, et al. Greener route for synthesis of aryl and alkyl-14H-dibenzo [aj] xanthenes using graphene oxide-copper ferrite nanocomposite as a recyclable heterogeneous catalyst. 2017;7 (1): 1-18.
54. Thirumalraj, et al. Highly sensitive electrochemical detection of palmatine using a biocompatible multiwalled carbon nanotube/poly-l-lysine composite. 2017;498:144-52.
55. Cao, et al. A new route to the considerable enhancement of glucose oxidase (GOx) activity: the simple assembly of a complex from CdTe quantum dots and GOx, and its glucose sensing. 2008; 14(31): 9633-40.
56. Dao,, et al. Insight into hydroxyl radical-mediated cleavage of caged methylene blue: the role of Fenton's catalyst for antimalarial hybrid drug activation. 2020;56 (80): 12017-20.
57. Żamojć, et al. The development of 1, 3-diphenylisobenzofuran as a highly selective probe for the detection and quantitative determination of hydrogen peroxide. 2017;51 (1): 38-46.
58. Das,et al. Piperine exhibits promising antibiofilm activity against Staphylococcus aureus by accumulating reactive oxygen species (ROS). 2022;204 (1): 1-11.
59. Rendueles O, Kaplan J B, Ghigo J M J Em. Antibiofilm polysaccharides. 2013; 15 (2): 334-46.
60. Pierscionek, et al. The effect of changing intraocular pressure on the corneal and scleral curvatures in the fresh porcine eye. 2007;91 (6): 801-03.
61. Yuan, et al. Near-Infrared Light-Triggered Nitric-Oxide-Enhanced Photodynamic Therapy and Low-Temperature Photothermal Therapy for Biofilm Elimination. ACS Nano 2020; 14 (3): 3546-62. doi: 10.1021/acsnano.9b09871 [published Online First: 2020 Feb. 19]
62. Ma C, et al. Enhancement of H2O2 decomposition efficiency by the co-catalytic effect of iron phosphide on the Fenton reaction for the degradation of methylene blue. Applied Catalysis B: Environmental 2019;259 doi: 10.1016/j.apcatb.2019.118015
63. Taverne, et al. (2013). Reactive oxygen species and the cardiovascular system. Oxidative medicine and cellular longevity, 2013.
64. Dickinson, et al. (2011). Chemistry and biology of reactive oxygen species in signaling or stress responses. Nature chemical biology, 7 (8), 504-511.
65. Han, et al. (2020). Biofilm microenvironment activated supramolecular nanoparticles for enhanced photodynamic therapy of bacterial keratitis. Journal of Controlled Release, 327, 676-687.
66. Ung, et al. (2019). The persistent dilemma of microbial keratitis: Global burden, diagnosis, and antimicrobial resistance. Survey of ophthalmology, 64 (3), 255-271.
67 Dua, et al. (1994). Corneal epithelial wound healing. The British journal of ophthalmology, 78 (5), 401.
68. Yonekawa, et al. (2014). Ocular blast injuries in mass-casualty incidents: the Marathon bombing in Boston, Massachusetts, and the fertilizer plant explosion in West, Texas. Ophthalmology, 121 (9), 1670-1676.
69. Memar, et al. (2018). Antimicrobial use of reactive oxygen therapy: current insights.

Infection and drug resistance, 11, 567.
70. Yin, et al. (2020). The antibacterial mechanism of silver nanoparticles and its application in dentistry. International journal of nanomedicine, 15, 2555.
71. Fong, et al. (2004). Clinical characteristics of microbial keratitis in a university hospital in Taiwan. American Journal of ophthalmology, 137 (2), 329-336.
72. Yao, et al. (2019). Reactive oxygen species (ROS)-responsive biomaterials mediate tissue microenvironments and tissue regeneration. Journal of Materials Chemistry B, 7 (33), 5019-5037.
73. Chuang, et al. Staphylococcus aureus ocular infection: methicillin-resistance, clinical features, and antibiotic susceptibilities. PLOS One 2012;8 (8): e42437. doi: 10.1371/journal.pone.0042437 [published Online First: 2012 Aug. 11] N M, J M, VU, et al. Unraveling genomic and phenotypic nature of multidrug-resistant (MDR) Pseudomonas aeruginosa VRFPA04 isolated from keratitis patient. Microbiol Res 2016;193:140-49. doi: 10.1016/j.micres.2016.10.002 [published Online First: 2016 Nov. 9].
74. Nie, et al., Pharmaceutics. 2022 December; 14 (12): 2635.
75. Wang, et al., ACS Appl. Mater. Interfaces 2022, 14, 41, 46324-46339.
75. Degn, et al., Bioact Mater, 2022 25:748-765

We claim:

1. An ocular composition comprising: one or more photoactive heterojunction materials alone or in combination with an enzyme capable of generating ROS upon contact with eye tears, in a pharmaceutically acceptable ocular carrier.

2. The composition of claim 1, wherein the pharmaceutically acceptable ocular carrier is a hydrogel.

3. The composition of claim 1 wherein the enzyme capable of generating ROS upon contact with eye tears is glucose oxidase and/or lactate oxidase.

4. The composition of claim 1, wherein the enzyme is glucose oxidase.

5. The composition of claim 1, wherein the enzyme is lactate oxidase.

6. The composition of claim 1, wherein the one or more photoactive heterojunction material is selected from the group consisting of Graphene oxide/copper ferrite nanoparticles (Go/CFN), Silver-titanium oxide (Ag—TiO₂), TiO₂/AgVO₃, Ag/TiO₂/cellulose, Alginate/Au—TiO₂, Lithium-titanate in the low-density polyethylene matrix (Li—TiO₂/LDPE), Cu—TiO₂, TiO₂/—Fe₂O₃, TiO2 doped with Bi (Bi—TiO2 group) and TiO₂ co-doped with urea and Bi (Urea, Bi—TiO₂ group; U,Bi—TiO₂), chitosan films containing melon/TiO₂ (CTS/MTiO₂), TiO₂ nanoparticles and graphene sheets (TiO₂/GSs), graphene oxide and cuprous oxide (rGO-Cu₂O), and Zinc oxide-selenium (ZnO—Se), optionally, wherein the photoactive heterojunction material comprises Go/CFN and/or shows strong light absorbance at 808 nm.

7. The composition of claim 1, wherein: (a) the hydrogel is made from a synthetic or naturally occurring polymer, a polysaccharides or glycosaminoglycan, optionally, wherein the polymer is a protein, or peptide, and/or (b) the hydrogel has antibacterial activity.

8. The composition of claim 7, wherein the hydrogel is made from an agent selected from the group consisting of poly & lysine, fluorenylmethoxycarbonyl (Fmoc)-diphenylalanine and Fmoc-capped short peptides bearing either lysine-rich or pyridinium groups, anthranilamide-based diphenylalanine peptide mimics; and naphthyl anthranilamide (NaA) capped amino acid based cationic peptide mimics.

9. The composition of claim 8, herein the hydrogel is made from an Fmoc-capped peptide selected from the group consisting of

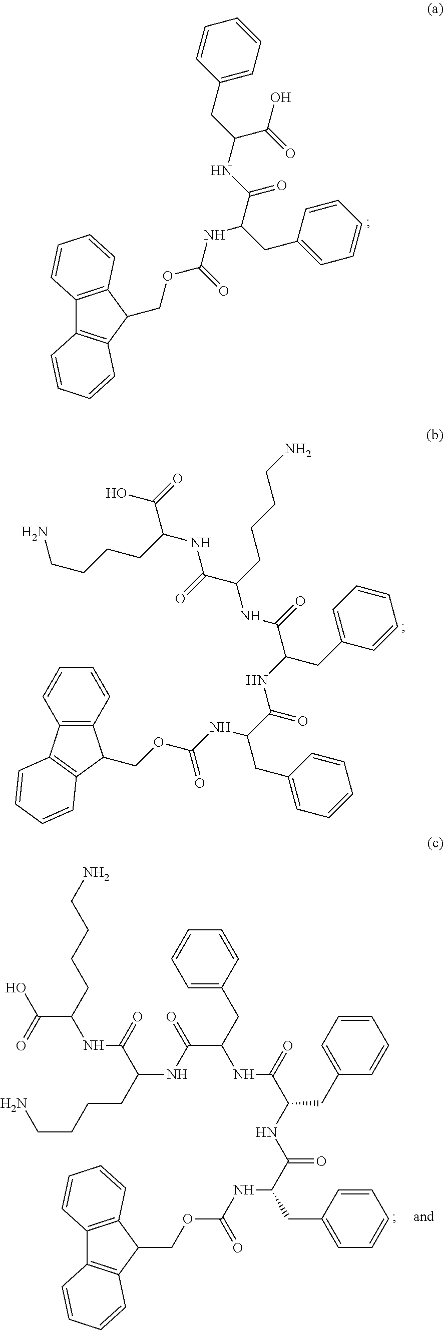 [SMILES: O=C(NC(Cc1ccccc1)C(=O)NC(Cc1ccccc1)C(=O)O)OCC1c2ccccc2-c2ccccc21 ; NCCCCC(NC(=O)C(CCCCN)NC(=O)C(Cc1ccccc1)NC(=O)C(Cc1ccccc1)NC(=O)OCC1c2ccccc2-c2ccccc21)C(=O)O ; NCCCCC(NC(=O)C(CCCCN)NC(=O)C(Cc1ccccc1)NC(=O)[C@H](Cc1ccccc1)NC(=O)[C@H](Cc1ccccc1)NC(=O)OCC1c2ccccc2-c2ccccc21)C(=O)O]

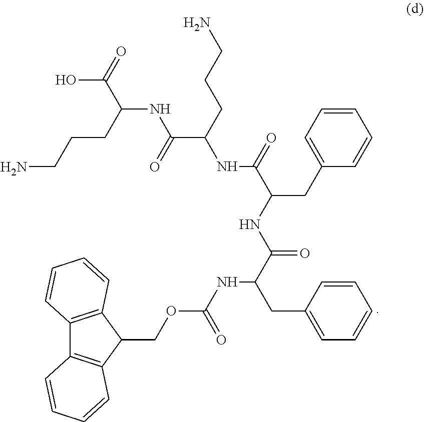 [SMILES: NCCCC(NC(=O)C(CCCN)NC(=O)C(Cc1ccccc1)NC(=O)C(Cc1ccccc1)NC(=O)OCC1c2ccccc2-c2ccccc21)C(=O)O]

10. The composition of claim 7, wherein the naphthyl anthranilamide (NaA) capped amino acid based cationic peptide or peptide mimic is selected from the peptides in FIG. 9C or 9D.

11. The composition of claim 1, wherein the hydrogel is a poly ε lysine based hydrogel, comprising following constitutional unit: 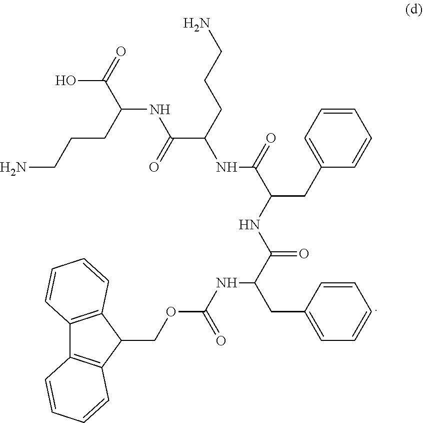
wherein, n is any natural number of 20 to 30, m is any natural number of 50 to 70.

12. The composition of claim 1, wherein the amount of photoactive material incorporated into the hydrogel range from about 50 μg to about 250 μg, preferably from about 80 μg to about 200 μg.

13. A formulation comprising the composition claim 1, in a form selected from the group consisting an ophthalmic gel (including contact lenses, such as bandage contact lenses (BCL)), an ocular wound bandage, and dosage forms application for treatment of corneal wound healing such as amniotic membrane (AM) bandages, bandage contact lenses (BCL), and collagen shields, optionally, wherein the formulation is not a liquid eyedrop which remains liquid following administration into the eye.

14. The formulation of claim 16, in the form of a soft or hard contact lens, or a bandage contact lens.

15. The formulation of claim 13 comprising a soft contact lens comprising crosslinked hydrogels including hydrophilic monomers selected from the group consisting of N-Vinylpyrrolidone, N,N-dimethylacrylamide, 2-hydroxyethyl methacrylate, hydroxyethyl acrylate, methacrylic acid and acrylic acid), crosslinked hydrogels containing silicone macromers and monomers, the formulation further comprises strengthening agents, ultraviolet light (UV) blockers, and/or tints.

16. A method of treating or preventing an ocular infection in a subject in need thereof, comprising contacting an eye of the subjection with the composition of claim 1, in combination with near infrared radiation (NIR).

17. The method of claim 16, wherein the NIR is provided by a light source 2 cm to about 8 cm, preferably from about 3 cm to about 5 cm, more preferably about 4 cm from the eye of the subject, and/or wherein the duration of treatment is from about 20 minutes to about 30 minutes daily for about 3 days to about 5 days.

18. The method of claim 16, wherein the ocular infection is caused by one or more organisms selected from the group of Staphylococcus, Pseudomonas, Streptococcus, Haemophilus influenza; Haemophilus ducreyi; Moraxella catarrhalis; Neisseria; Chlamydia Mycobacterium Bordetella pertussis; Campylobacter jejuni; Legionella pneumophila; Bacteroides bivius; Clostridium perfringens; Peptostreptococcus species; Borrelia burgdorferi; Mycoplasma pneumoniae; Treponema pallidum; Ureaplasma urealyticum; Toxoplasma; and Nosema.

19. The method of claim 23, wherein the ocular infection is caused by S. aureus, P. aeruginosa, Methicillin-resistant Staphylococcus aureus and multi-drug resistant Pseudomonas aeruginosa.

20. The method of claim 17, wherein: (a) the subject does not suffer from dry eyes and/or has not received refractive surgery; and/or (b) contacting the eye with the formulation and NIR produces reactive oxygen species in an effective amount to inhibit bacterial growth and/or promote bacterial cell death.
